(19) United States
(12) Patent Application Publication
(10) Pub. No.: US 2015/0216843 A1
(43) Pub. Date: Aug. 6, 2015

(54) INHIBITION OF CXCR4 SIGNALING IN CANCER IMMUNOTHERAPY

(71) Applicant: CAMBRIDGE ENTERPRISE LIMITED, Cambridge (GB)

(72) Inventor: Douglas Fearon, Lloyd Harbor, NY (US)

(73) Assignee: Cambridge Enterprise Limited

(21) Appl. No.: 14/620,463

(22) Filed: Feb. 12, 2015

Related U.S. Application Data
(63) Continuation of application No. PCT/IB2014/063706, filed on Aug. 5, 2014.
(60) Provisional application No. 62/023,909, filed on Jul. 13, 2014.
(60) Provisional application No. 62/018,837, filed on Jun. 30, 2014.

(30) Foreign Application Priority Data
Aug. 5, 2013 (GB) 1313983.7
Sep. 27, 2013 (GB) 1317213.5
Nov. 18, 2013 (GB) 1320329.4

(51) Int. Cl. A61K 31/395; A61K 31/7028; A61K 31/713; A61K 39/395; A61K 47/48

(52) U.S. Cl. CPC A61K 31/395; A61K 39/39558; A61K 47/48215; A61K 31/713; A61K 31/7028

(57) ABSTRACT

The inventions describes a method for increasing effector T cell accumulation in cancer cell-containing sites of a tumor, comprising administering to a subject in need thereof a pharmaceutically effective amount of an inhibitor of CXCR4 signaling.

The present invention is concerned with therapy of tumors. In particular, the invention is concerned with reducing or preventing immune suppression and increasing T cell recruitment and accumulation in the cancerous tumor microenvironment, in order to overcome the exclusion and death of CD3+ T cells, and preferably CD3+ effector T cells from the tumor and the suppression of anti-tumor T-cell activity.

### INTRODUCTION

Immunotherapy of cancer has made recent progress by focusing on overcoming T cell immunological checkpoints with blocking monoclonal antibodies to CTLA-4 and the PD-1/PD-L1 receptor/ligand pair, leading to noteworthy results in cancer patients (1-6). Many patients, however, did not respond to these immunological checkpoint antagonists for reasons that are not understood. For example, patients with pancreatic ductal adenocarcinoma (PDA), the fourth most common cause of cancer-related deaths in the United States, had no objective responses to α-CTLA-4 (7) or α-PD-L1 monoclonal antibodies (5).

Cancer is the second leading cause of death in the United States, exceeded only by heart disease. Despite recent advances in cancer diagnosis and treatment, surgery and radiotherapy may be curative if a cancer is found early, but current drug therapies for metastatic disease are mostly palliative and seldom offer a long-term cure. Even with new chemotherapies entering the market, the need continues for new drugs effective in monotherapy or in combination with existing agents as first line therapy, and as second and third line therapies in treatment of resistant tumors.

Cancer cells are by definition heterogeneous. For example, within a single tissue or cell type, multiple mutational ‘mechanisms’ may lead to the development of cancer. As such, heterogeneity frequently exists between cancer cells taken from tumors of the same type that have originated in different individuals and even between cancer cells from different regions of a tumor in a single individual. Frequently observed mutational ‘mechanisms’ associated with some cancers may differ between one tissue type and another (e.g., frequently observed mutational ‘mechanisms’ leading to colon cancer may differ from frequently observed ‘mechanisms’ leading to leukemias). It is therefore often difficult to predict whether a particular cancer will respond to a particular chemotherapeutic agent. (Cancer Medicine, 5th Edition, Bast et al. eds., B. C. Decker Inc., Hamilton, Ontario).

Recent efforts in treating cancer focus on targeted therapeutics or treatments that specifically inhibit vital signaling pathways. However, drug resistance and cancer progression invariably develop. Accordingly, new compounds and methods for treating cancer are needed. The present invention addresses these needs.

CXCL12 is a chemokine that localizes to human PDA. However, there are mixed reports linking CXCL12 to cancer. It has been suggested that antagonizing CXCL12, or its receptor, CXCR4, increases T-cell trafficking across the blood-brain barrier and improves survival rates from West Nile virus disease (McCandless et al.) and it has been speculated that anti-CXCL12 therapy might be useful for the treatment of ovarian cancer, because CXCL12 inhibition leads to a reduction in FoxP3+ regulatory T-cells in ovarian tumors (Righi et al., Cancer Res. 2011 Aug. 15; 71(16):5522-34). However, the art concerning CXCL12 is unclear, with some reports indicating that CXCL12 expression impairs immune control in a tumor established with the B16 tumor cell line (20; Righi et al.; Vianello et al., J Immunol. 2006 Mar. 1; 176(5):2902-14), while other studies indicate the CXCL12 expression enhances immune control (Nomura et al., Int J Cancer. 2001 Mar. 1; 91(5):597-606; Fushimi et al., Cancer Res. 2006 Apr. 1; 66(7):3513-22; Williams et al., Mol Cancer. 2010 Sep. 17; 9:250; and Dannussi-Joannopoulos et al., Blood. 2002 Sep. 1; 100(5):1551-8). Thus, it is not clear whether CXCL12 has any role in the development of cancer.

The present invention addresses the continued need to improve and develop new cancer treatments.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject.

The present invention relates to a method of inhibiting T cell exclusion in a tumor, wherein the method comprises administering to a patient a pharmaceutically effective amount of a CXCR4 signaling inhibitor wherein the CXCR4 signaling inhibitor increases the proximity or the frequency of the T-cells among the cancer cells contained in the tumor.

In preferred embodiments, the method of the present invention increases both the proximity and the frequency of T-cells among the cancer cells contained in the tumor. In further preferred embodiments, the proximity of the T cells among the cancer cells is increased by at least 2 fold (distance between cancer cell and nearest T cell is decreased by 2 fold), 3 fold (distance between cancer cell and nearest T cell is decreased by 3 fold), 4 fold (distance between cancer cell and nearest T cell is decreased by 4 fold) or 5 fold (distance between cancer cell and nearest T cell is decreased by 5 fold). In further preferred embodiments, the frequency of the T cells among the cancer cells is increased by at least 20%, at least 30%, at least 40%, at least 50%, at least 60%, at least 70%, or at least 80%. In further preferred embodiments, the T-cells are CD3+ effector T-cells.

In a further preferred embodiment, the method increases the sensitivity of the cancer cells to the host immune responses or reduces immune suppression in the tumor. Immune suppression can be caused by either T-cell apoptosis or ligation of CXCR4 on T cells, which may cause T-cell apoptosis. In a further preferred embodiment, the relief of T-cells from immune suppression allows them to cause apoptosis of cancer cells.

In an even further preferred embodiment, the method increases cancer cell recognition within the tumor. In an even further preferred embodiment, the method inhibits cancer cell growth. In an even further preferred embodiment, the method eliminates cancer cells. In an even further preferred embodiment, the method reduces tumor mass. In an even further embodiment, the tumor mass is comprised of p53+ cancer cells.

In a further preferred embodiment, the tumor comprises FAP+ stromal cells. In a further preferred embodiment the tumor is resistant to immunotherapy.

In preferred embodiments, the tumor is an adenocarcinoma, sarcoma, skin cancer, melanoma, bladder cancer, brain cancer, breast cancer, uterine cancer, ovarian cancer, prostate cancer, lung cancer, colorectal cancer, cervical cancer, liver cancer, head and neck cancer, esophageal cancer, pancreas cancer, pancreatic ductal adenocarcinoma (PDA), renal cancer, stomach cancer, multiple myeloma or cerebral cancer.

In preferred embodiments, the CXCR4 signaling inhibitor is a CXCL12 antagonist. In further preferred embodiments, the CXCL12 antagonist is an anti-CXCL12 antibody. One example of an anti-CXCL12 antibody includes, but is not limited to an anti-SDF-1 antibody. Examples of such a CXCL12 antagonist, can be, but are not limited to RNA oligonucleotide NOX-A12 or Tannic acid or any other chemical that blocks the interaction of CXCL12 with CXCR4.

In other preferred embodiments, the CXCR4 signaling inhibitor is a CXCR4 antagonist. In further preferred embodiments, the CXCR4 antagonist is an anti-CXCR4 antibody.

In further preferred embodiments, the CXCR4 antagonist is BMS-936564/MDX-1338, LY2510924, 1,1′-[1,4-phenylenebis(methylene)]bis[1,4,8,11-tetraazacyclotetradecane] (AMD3100; Plerixafor), N,N-dipropyl-N-[4-({[(1H-imidazol-2-yl)methyl)benzyl][(1-methyl-1H-imidazol-2-yl)methyl]amino]methyl)benzyl]-N-methylbutane-1,4-diamine tri(2R,3R)-tartrate (KRH-3955), ([5-(4-methyl-1-piperazinyl)-2-({methyl[(8S)-5,6,7,8-tetrahydro-8-quinolinyl]amino}methyl)imidazo[1,2-a]pyridin-3-yl]methanol) (GSK812397), or N-(1H-benzimidazol-2-ylmethyl)-N′-(5,6,7,8-tetrahydroquinolin-8-yl)butane-1,4-diamine (AMD11070).

In further preferred embodiments, the method also comprises administering a PD-1 signaling inhibitor. In further preferred embodiments, the PD-1 signaling inhibitor is a PD-1 antagonist. In further preferred embodiments, the PD-1 antagonist is an anti-PD-1 antibody. In further preferred embodiments, the PD-1 signaling inhibitor is a PDL-1 antagonist. In further preferred embodiments, the PDL-1 antagonist is an anti-PDL-1 antibody. Thus, in preferred embodiments, the CXCR4 signaling inhibitor of the present invention is administered in combination with a PD-1 signaling inhibitor, and preferably to a PD-1 antagonist, including for example, an anti-PD-1 antibody, or a PD-L1 antagonist, including for example, an anti-PD-L1 antibody.

In further embodiments, the method also comprises administering a CTLA-4 antagonist. In further embodiments, the CTLA-4 antagonist is an anti-CTLA-4 antibody. Thus, in preferred embodiments, the CXCR4 signaling inhibitor of the present invention is administered in combination with a CTLA-4 antagonist, including for example, an anti-CTLA-4 antibody.

In further embodiments, the method also comprises administering a TIM-3 antagonist. In even further embodiments, the TIM-3 antagonist is an anti-TIM-3 antibody. Thus, in preferred embodiments, the CXCR4 signaling inhibitor of the present invention is administered in combination with a TIM-3 antagonist, including for example, an anti-TIM-3 antibody.

In further embodiments, the method also comprises administering a LAG3 antagonist. In even further embodiments, the LAG3 antagonist is an anti-LAG3 antibody. Thus, in preferred embodiments, the CXCR4 signaling inhibitor of the present invention is administered in combination with a LAG3 antagonist, including for example, an anti-LAG3 antibody.

In further embodiments, the method also comprises administering a checkpoint antagonist. Thus, in preferred embodiments, the CXCR4 signaling inhibitor of the present invention is administered in combination with a checkpoint antagonist, including for example, an antibody directed to a checkpoint protein.

In other preferred embodiments, the method also comprises administering an agonist to a T cell co-receptor. Examples of such agonists to T cell co-receptor, include, but are not limited an agonistic antibody to a T cell co-receptor. Examples of such T cell co-receptors, include, but are not limited to, 4-1BB (CD137) and ICOS (CD278). Thus, in preferred embodiments, the CXCR4 signaling inhibitor of the present invention is administered in combination with an agonist to a T cell co-receptor, and preferably to an agonistic antibody to a T cell co-receptor, and even more preferably, an agonistic antibody to 4-1BB (CD137) or ICOS (CD278).

In other preferred embodiments, the PD-1 signaling inhibitor, PD-1 antagonist, the anti-PD-1 antibody, the PD-L1 antagonist, the anti-PD-L1 antibody, the CTLA-4 antagonist, the anti-CTLA-4 antibody, the TIM-3 antagonist, the anti-TIM-3 antibody, the LAG3 antagonist, the anti-LAG3 antibody, the T cell co-receptor agonist, the T cell co-receptor agonistic antibody, the agonistic antibody to 4-1BB (CD137), the agonistic antibody to ICOS (CD278) and/or the checkpoint antagonist acts synergistically with the CXCR4 signaling inhibitor.

In other preferred embodiments, the method also comprises administering other anti-cancer therapies. In these embodiments, other anti-cancer therapies include, but are not limited to: chemotherapeutic agents, radiation therapy, cancer therapy, immunotherapy, or cancer vaccines. Examples of such immunotherapies include, but not limited to adoptive T cell therapies or cancer vaccine preparations designed to induce T lymphocytes to recognize tumor cells.

In other preferred embodiments, the cancer vaccine recognizes one or more tumor antigens expressed on the cancer cells. Examples of such tumor antigens include, but are not limited to MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2(HOM-MEL-40), SSX-3, SSX-4, SSX-5, SCP-I and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS, tyrosinase related proteins, TRP-1, or TRP-2.

In other preferred embodiments, the anti-cancer therapy includes, but is not limited to: aspirin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGF/VEGF receptors, platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; inhibitors of PI-3 kinase, including PI-3 kinasedelta; and anti-virals such as oseltamivir phosphate, Amphotericin B, and palivizumab.

In other preferred embodiments, the CXCR4 signaling inhibitor and the PD-1 signaling inhibitor and/or the anti-cancer therapy is administered simultaneously, separately, or sequentially.

In further preferred embodiments, the patient is a human. In other preferred embodiments, the “patient” or “subject suitable for treatment” may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human. In other embodiments, non-human mammals, especially mammals that are conventionally used as models for demonstrating therapeutic efficacy in humans (e.g. murine, primate, porcine, canine, or rabbit animals) may be employed.

In embodiments of the invention, the increase in T-cell accumulation is effective to reduce the growth rate and immune evasion of a tumor. We have observed that recruitment of CD3+ T-cells to the cancer cell-containing sites of the tumor causes tumor growth to be impeded, as a result of immunological regulation of the tumor.

In another aspect, there is provided a method for treating a cancer comprising administering to a subject in need thereof a pharmaceutically effective amount of an inhibitor of CXCR4 signaling. In one preferred embodiment, the cancer is a pancreatic tumor. In one further embodiment, the pancreatic tumor is a pancreatic ductal adenocarcinoma (PDA).

In another aspect, there is provided a use of an inhibitor, such as Plerixafor, of CXCR4 signaling for increasing the proximity of T-cells to cancer cells of a tumor. One mechanism of increasing the proximity of T-cells to cancer cells is by decreasing the sensitivity of T-cells to FAP+ stromal cell-derived CXCL12 which coats the cancer cells within the tumor, which in turn would interact with CXCR4 on T cells. A second mechanism is by reducing immune suppression in a cancerous tumor comprised of FAP+ stromal cells in an individual.

In another aspect, there is provided the use of an inhibitor of CXCR4 signaling for the treatment of a tumor. In one embodiment, the tumor is a pancreatic tumor, and an even further embodiment, the tumor is a pancreatic ductal adenocarcinoma (PDA). In another aspect, the invention provides a method of promoting T cell infiltration into cancerous tumor tissue containing FAP+ stromal cells by administering a CXCR4 signaling inhibitor to the individual. In preferred embodiments, the CXCR4 signaling inhibitor is Plerixafor.

In another preferred embodiment, the invention provides the use of a CXCR4 signaling inhibitor in the manufacture of a medication for reducing immune suppression in a tumor, preferably, a tumor comprised of FAP+ stromal cells. The FAP+ stromal cells express CXCL12, thereby coating the cancer cells within the tumor with CXCL12. This coating then mediates the exclusion of CXCR4-expressing T cells by causing their apoptosis. This reaction accounts for the presence of T cells almost exclusively in the stromal regions of the tumor and not in the vicinity of or amongst cancer cells. Accordingly, the use of a CXCR4 signaling inhibitor decreases the exclusion of T cells within the cancer (e.g., increasing the proximity of T cells to cancer cells within the tumor) and leads to eventual cancer cell death.

### BRIEF DESCRIPTION OF THE FIGURES

Embodiments are illustrated by way of example and not limitation in the figures of the accompanying drawings, in which like references indicate similar elements and in which:

FIG. 1 shows the increase in PDA volume (mean±SEM) following treatment of KPC mice with anti-PD-L1 (n=6), anti-CTLA-4 (n=6) or control (n=4) antibodies as measured by ultrasound.

FIG. 2 shows the induction by different types of pancreas-derived cells of IFN-gamma-secretion by splenic CD8+ T cells from various donors was measured by ELISpot assay. *P<0.05, 7 ***P<0.001; (left) and (middle), n≧8; (right) Mann-Whitney test, n=4.

FIG. 3 shows qRT-PCR of Fap mRNA in tumors excised on day 6 from DTR BAC transgenic mice with PDA given Diphtheria toxin (DTx) or PBS (PBS n=5; DTx n=7).

FIG. 4 shows (left) tumor volumes in mice with or without DTR BAC transgene and treated with DTx or PBS (PBS n=6; DTx n=8; DTx to non BAC DTR transgenic n=4) and (right) tumor volumes in BAC DTR transgenic mice with PDA administered control IgG or CD4- and CD8-depleting antibodies prior to and during treatment with DTx or PBS (αaCD4/8+PBS n=3; α-CD4/8+DTx n=5; isotype IgG+DTx, n=5).

FIG. 5 shows tumor volumes in BAC DTR transgenic mice with PDA administered anti-CTLA-4 or anti-PD-L1 during treatment with DTx or PBS (DTx n=13, which represent all DTx-treated mice accumulated throughout the course of this study; anti-CTLA-4+DTx n=6; anti-PD-L1+DTx n=4).

FIG. 6 shows “Waterfall” plots that demonstrate the tumor volume changes in individual mice. *P<0.05, **P<0.01.

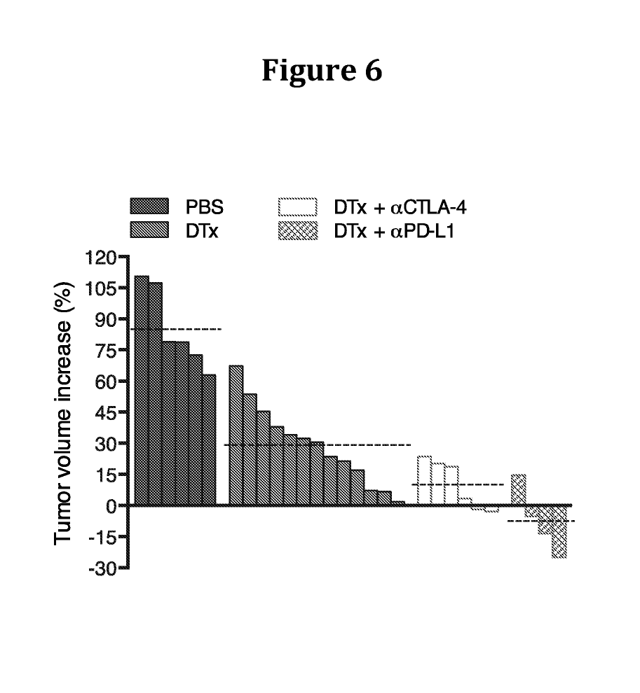

FIG. 7 shows qRT-PCR measurements of FACS-purified cells from (mean of) three tumors. Cxcl12 mRNA is more highly expressed by FAP+ cells than CD11b+ cells or PDA/PanIN cells (CD11b−/CD45−/FAP−).

FIG. 8 shows tumor volumes measured in (left) PDA-bearing mice following implantation of continuous infusion osmotic pumps containing PBS or AMD3100. Some had been pre-treated with control IgG or depleting CD4 and CD8 antibodies (PBS n=5, AMD3100+isotype IgG n=6, AMD3100+anti-CD4/8 n=4); and (right) tumor volumes measured in PDA-bearing mice implanted with continuous infusion osmotic pumps containing AMD3100 (high dose) that were also given control IgG, or anti-CTLA-4 or anti-PD-L1 (AMD3100 high+isotype IgG (n=6), AMD3100 high+anti-CTLA-4 (n=4), AMD3100 high+anti-PD-L1 (n=7)).

FIG. 9 shows waterfall plots demonstrating the changes in tumor volumes in individual mice from FIG. 8.

FIG. 10 Confocal micrographs of mouse pancreatic tumor sections from mice treated for 24 h with (A) anti-PD-L1, (B) AMD3100 or (C) both AMD3100 and anti-PD-L1, and stained with antibodies to p53 to demonstrate cancer cells, and anti-CD3epsilon to demonstrate T cells. There is exclusion of T cells from the tumor region containing p53+ cancer cells, even after treatment with anti-PD-L1. This exclusion is overcome by AMD3100, and the combination with anti-PD-L1 causes loss of cancer cells. Panel D is a histogram showing the distance from each cancer cell to the nearest T cell in tumors taken from mice treated for 24 h with PBS, anti-PD-L1, AMD3100, and AMD3100+anti-PD-L1, respectively.

FIG. 11 shows confocal micrographs of mouse pancreatic tumor sections from mice treated for 6 days with PBS or AMD3100 and anti-PD-L1, and stained with antibodies to p53 to demonstrate cancer cells (D). Sections also were stained with Ki67 antibody to identify cells in cell cycle (E), and CK19 and CD45 antibodies to identify pancreatic epithelial cells and inflammatory cell (F).

### DETAILED DESCRIPTION OF THE INVENTION

### A. Definitions

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, N.Y.; Ausubel et al., Short Protocols in Molecular Biology (1999) 4^{th} ed., John Wiley & Sons, Inc.), which are incorporated herein by reference.

As used herein, “T cell exclusion” in a tumor is defined as those tumor evasion mechanisms known in the art where effector CD3+ T cell subsets are prevented from recruitment and accumulation at the cancerous tumor microenvironment.

As used herein, “T cells” or “CD3+ T cells” are defined as those lymphocyte lineage cells that express the cell surface marker CD3, which includes CD4+ T cells, CD8+ T cells, and Foxp3+ regulatory T cells. “Effector CD3+ T cells” are defined as those mature T cell population groups that assist with the activity of other immune cells by releasing T cell cytokines or have direct cytotoxic function. Such cells include CD4+ T cells, CD8+ T cells, and Foxp3+ regulatory T cells.

In the present invention, a “CXCR4 signaling inhibitor” is an exogenous factor, such as a pharmaceutical compound or molecule, that inhibits or prevents the activation of CXCR4 by its ligand C—X—C motif ligand 12 (CXCL12) and thereby blocks or inhibits CXCR4 signaling in cells within the cancerous tumor.

Suitable CXCR4 signaling inhibitors may be identified using standard in vitro or ex vivo CXCL12/CXCR4 ligation assays, such as chemotaxis or increased free intracellular Ca²⁺. For example, the absence of rapid, transient increases in free intracellular Ca²⁺ when CXCR4 on a cell surface is exposed to CXCL12 may be indicative of the presence of a CXCR4 signaling inhibitor.

Preferred examples of a CXCR4 signaling inhibitor includes, but is not limited to, a CXCR4 antagonist and/or a CXCL12 antagonist.

In the present invention, a “CXCR4 antagonist” is defined as a molecule that inhibits CXCR4 signaling by binding to or interacting with CXCR4 to prevent or inhibit the binding and/or activation of CXCR4 by CXCL12, thereby inhibiting CXCR4 signaling. Preferred examples of a CXCR4 antagonist, include, but are not limited to an anti-CXCR4 antibody, examples of which are well known in the art. For example, preferred anti-CXCR4 antibodies include, but are not limited to BMS-936564/MDX-1338 (Kuhne et al (2013) Clin Cancer Res 19(2) 357-366).

Additionally, CXCR4 antagonists include peptides, such as LY2510924 (Eli Lilly) or small organic compounds, such as 1,1′-[1,4-phenylenebis(methylene)]bis[1,4,8,11-tetraazacyclotetradecane] (AMD3100; Plerixafor), N,N-dipropyl-N-[4-({[(1H-imidazol-2-yl)methyl)benzyl][(1-methyl-1H-imidazol-2-yl)methyl]amino]methyl)benzyl]-N-methylbutane-1,4-diamine tri(2R,3R)-tartrate (KRH-3955), ([5-(4-methyl-1-piperazinyl)-2-({methyl[(8S)-5,6,7,8-tetrahydro-8-quinolinyl]amino}methyl)imidazo[1,2-a]pyridin-3-yl]methanol) (GSK812397; Jenkinson et al., Antimicrob. Agents Chemother. 2010, 54(2):817), and N′-(1H-benzimidazol-2-ylmethyl)-N′-(5,6,7,8-tetrahydroquinolin-8-yl)butane-1,4-diamine (AMD11070; Moyle et al Clin. Infect. Dis. 48:798-805)).

In the present invention, a “CXCL12 antagonist” is defined as a molecule that inhibits CXCR4 signaling by binding to or inhibiting CXCL12 from binding and/or activating CXCR4, thereby inhibiting CXCR4 signaling. CXCL12 may, for example, be produced by stromal cells in the cancerous tumor that express fibroblast activation protein (FAP). Preferred examples of a CXCL12 antagonist, include, but are not limited to an anti-CXCL12 antibody, which are well known in the art. Examples of such anti-CXCL12 antibodies, include, but are not limited to an anti-CXCL12 antibody from R&D Systems (MAB310) or SDF-1 antibody. Other examples of CXCL12 antagonists include, but are not limited to, NOX-A12.

Other suitable CXCR4 and CXCL12 antagonists include non-antibody specific binding molecules, such as adnectins, affibodies, avimers, anticalins, tetranectins, DARPins, mTCRs, engineered Kunitz-type inhibitors, nucleic acid aptamers and spiegelmers, peptide aptamers and cyclic and bicyclic peptides (Ruigrok et al Biochem. J. (2011) 436, 1-13; Gebauer et al Curr Opin Chem Biol. (2009)(3):245-55). Suitable specific binding molecules for use as CXCR4 and CXCL12 antagonists may be generated using standard techniques.

CXCR4 signaling is mediated by activation of phosphoinositide 3-kinases. Other suitable CXCR4 signaling inhibitors include PI 3-kinase inhibitors, for example inhibitors of p110 delta or p110 gamma isoforms of PI3K.

Suitable PI3K inhibitors include 5-fluoro-3-phenyl-2-([S)]-1-[9H-purin-6-ylamino]-propyl)-3H-quinazolin-4-one (CAL-101); acetic acid (1S,4E,10R,11R,13S,14R)-[4-diallylaminomethylene-6-hydroxy-1-methoxymethyl-10,13-dimethyl-3,7,17-trioxo-1,3,4,7,10,11,12,13,14,15,16,17-dodecahydro-2-oxa-cyclopenta[a]phenanthren-11-yl ester (PX-866) and (S)-3-(1-((9H-purin-6-yl)amino)ethyl)-8-chloro-2-phenylisoquinolin-1(2H)-one (IPI-145).

Other suitable PI 3-kinase inhibitors are well known in the art.

In the present invention, term “antibody,” refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. Examples of molecules which are described by the term “antibody” in this application include, but are not limited to: single chain Fvs (sdFvs), Fab fragments, Fab′ fragments, F(ab′)2, disulfide linked Fvs (sdFvs), Fvs, and fragments comprising or alternatively consisting of, either a VL or a VH domain. The term “single chain Fv” or “scFv” as used herein refers to a polypeptide comprising a VL domain of antibody linked to a VH domain of an antibody.

Antibodies of the invention include, but are not limited to, monoclonal, multispecific, bi-specific, human, humanized, mouse, or chimeric antibodies, single chain antibodies, Fab fragments, F(ab′) fragments, antiidiotypic (anti-Id) antibodies (including, e.g., anti-Idantibodies to antibodies of the invention), and epitope-binding fragments of any of the above. The immunoglobulin molecules of the invention can be of any type (e.g., IgG, IgE, IgM, IgD, IgA and IgY), class (e.g., IgG1, IgC2, IgG3, IgG4, IgA1 and IgA2) or subclass of immunoglobulin molecule.

In the present invention, a “PD-1 signaling inhibitor” is an exogenous factor, such as a pharmaceutical compound or molecule that inhibits or prevents the activation of PD-1 by its ligand PD-L1 and thereby blocks or inhibits PD-1 signaling in cells within the cancerous tumor. A PD-1 signaling inhibitor is defined broadly as any molecule that prevents the negatively regulation by PD-1 of T-cell activation.

Preferred examples of a PD-1 signaling inhibitor includes, but is not limited to, a PD-1 antagonist and/or a PD-L1 antagonist.

In the present invention, a “PD-1 antagonist” is defined as a molecule that inhibits PD-1 signaling by binding to or interacting with PD-1 to prevent or inhibit the binding and/or activation of PD-1 by PD-L1, thereby inhibiting PD-1 signaling and/or enhancing T cell activation. Preferred examples of a PD-1 antagonist, include, but are not limited to an anti-PD-1 antibody which are well known in the art. See, Topalian, et al. NEJM 2012.

In the present invention, a “PD-L1 antagonist” is defined as a molecule that inhibits PD-1 signaling by binding to or inhibiting PD-L1 from binding and/or activating PD-1, thereby inhibiting PD-1 signaling and/or enhancing T cell activation. Preferred examples of a PD-L1 antagonist, include, but are not limited to an anti-PD-L1 antibody which are well known in the art. See, Brahmer, et al. NEJM 2012.

In preferred embodiments, the CXCR4 signaling inhibitor of the present invention, whether it be a CXCR4 antagonist (for example an anti-CXCR4 antibody), or a CXCL12 antagonist (for example, an anti-CXCL12 antibody) has synergistic activity with a PD-1 signaling inhibitor of the present invention. In preferred embodiments, the CXCR4 signaling inhibitor is for example, AMD3100, BMS-936564/MDX-1338, AMD11070, or LY2510924 and has synergistic activity with a PD-1 signaling inhibitor of the present invention, such as, for example, an anti-PD-1 antibody or an anti-PD-L1 antibody.

In the present invention, a “CTLA-4 antagonist” is defined as a molecule that inhibits CTLA-4 signaling by binding to or inhibiting CTLA-4 from binding and/or activating to B7 molecules, known in the art to be present on antigen-presenting cells, thereby preventing interactions of B7 molecules with the co-stimulatory molecule CD28, and inhibiting T-cell function. Preferred embodiments of a CTLA-4 antagonist, include, but are not limited to anti-CTLA-4 antibodies.

In preferred embodiments, the CXCR4 signaling inhibitor of the present invention, whether it be a CXCR4 antagonist (for example an anti-CXCR4 antibody), or a CXCL12 antagonist (for example, an anti-CXCL12 antibody) has synergistic activity with a CTLA-4 antagonist of the present invention. In preferred embodiments, the CXCR4 signaling inhibitor is for example, AMD3100, BMS-936564/MDX-1338, AMD11070, or LY2510924 and has synergistic activity with a CTLA-4 antagonist of the present invention, such as, for example, an anti-CTLA-4 antibody.

In the present invention, a “LAG3 antagonist” is defined as a molecule that inhibits LAG3 signaling by binding to or inhibiting LAG3 from binding and/or activating MHC molecules and any other molecule, known in the art to be present on antigen-presenting cells, thereby preventing LAG3 interactions and promoting T-cell function. Preferred embodiments of a LAG3 antagonist, include, but are not limited to anti-LAG3 antibodies.

In preferred embodiments, the CXCR4 signaling inhibitor of the present invention, whether it be a CXCR4 antagonist (for example an anti-CXCR4 antibody), or a CXCL12 antagonist (for example, an anti-CXCL12 antibody) has synergistic activity with a LAG3 antagonist of the present invention. In preferred embodiments, the CXCR4 signaling inhibitor is for example, AMD3100, BMS-936564/MDX-1338, AMD11070, or LY2510924 and has synergistic activity with a LAG3 antagonist of the present invention, such as, for example, an anti-LAG3 antibody.

In the present invention, a “TIM-3 antagonist” is defined as a molecule that inhibits the CD8+ and CD4+ Th1-specific cell surface protein, TIM-3, which, when ligated by galectin-9, for example, causes T cell death. Preferred embodiments of a TIM-3 antagonist, include, but are not limited to anti-TIM-3 antibodies that block interaction with its ligands.

In preferred embodiments, the CXCR4 signaling inhibitor of the present invention, whether it be a CXCR4 antagonist (for example an anti-CXCR4 antibody), or a CXCL12 antagonist (for example, an anti-CXCL12 antibody) has synergistic activity with a TIM-3 antagonist of the present invention. In preferred embodiments, the CXCR4 signaling inhibitor is for example, AMD3100, BMS-936564/MDX-1338, AMD11070, or LY2510924 and has synergistic activity with a TIM-3 antagonist of the present invention, such as, for example, an anti-TIM-3 antibody.

In the present invention, a PD-1 antagonist, a CTLA-4 antagonist, a TIM-3 antagonist, and a LAG3 antagonist are T-cell checkpoint antagonists. Other examples of checkpoint antagonists are well known in the art. Blocking CXCR4 with any CXCR4 signaling inhibitor, leads to the uncovering of the anti-cancer effects of the T cell checkpoint antagonists.

In preferred embodiments, the CXCR4 signaling inhibitor of the present invention, whether it be a CXCR4 antagonist (for example an anti-CXCR4 antibody), or a CXCL12 antagonist (for example, an anti-CXCL12 antibody) has synergistic activity with a checkpoint antagonist of the present invention. In preferred embodiments, the CXCR4 signaling inhibitor is for example, AMD3100, BMS-936564/MDX-1338, AMD11070, or LY2510924 and has synergistic activity with a checkpoint antagonist of the present invention and those known in the art.

In the present invention, a “T cell co-receptor” is a cell surface receptor that binds to ligands on antigen-presenting cells that are distinct from the peptide-MHC complex that engages the T cell receptor. Ligation of T cell co-receptors enhance the antigen-specific activation of the T cell by recruiting intracellular signaling proteins (e.g., NFkappaB and PI3-kinase) inside the cell involved in the signaling of the activated T lymphocyte. Preferred embodiments of a T cell co-receptor antagonist, include, but are not limited to anti-T cell co-receptor antibodies, such as, for example, antibodies directed to 4-1BB (CD137) and ICOS (CD278).

In preferred embodiments, the CXCR4 signaling inhibitor of the present invention, whether it be a CXCR4 antagonist (for example an anti-CXCR4 antibody), or a CXCL12 antagonist (for example, an anti-CXCL12 antibody) has synergistic activity with a T cell co-receptor antagonist of the present invention. In preferred embodiments, the CXCR4 signaling inhibitor is for example, AMD3100, BMS-936564/MDX-1338, AMD11070, or LY2510924 and has synergistic activity with a T cell co-receptor antagonist of the present invention, such as, for example, an anti-T cell co-receptor antibody, for example, an anti-4-1BB (CD137) antibody or an anti-ICOS (CD278) antibody.

In the present invention, a “tumor” is defined as a population of heterogeneous cells, collectively forming a mass of tissue in a subject resulting from the abnormal proliferation of malignant cancer cells. In some preferred embodiments, the tumor may comprise of p53+ (Gene ID; 2191, reference sequence NP_{—}004451.2 GI:16933540) cancer cells. Thus, a “tumor” will contain both normal or “non-cancerous” cells and “cancer” or “cancerous” cells. A tumor typically comprises or is associated with p53+ and/or FAP+ stromal cells and/or inflammatory/immune cells. The cancer cells are often grouped together in “nests”, separated by stromal regions containing extracellular matrix (e.g., collagen), immune cells and FAP+ fibroblastic cells.

The presence of FAP+ stromal cells in a cancerous tumour may be identified using routine techniques, including protein based methods, such as fluorescence microscopy and immunohistology or nucleic acid based methods, such as RT-PCR. Kraman et al. Science. 330, 827-30 (2010).

In the present invention, “proximity” is defined as the distance between the CD3+ T-cells, and even more preferably effector CD3+ T-Cells, and the cancer cells within a tumor. For example, one way to measure “proximity” is to cross-section the tumor, such as a PDA tumor, and then stain the tumor with a cancer detecting antibody, such as anti-p53 (loss-of-heterozygosity at the p53 locus cancer cells are p53+) and anti-CD3epsilon (T cells are +). The section is then subjected to ARIOL scanning. An instrument then evaluates the image, and calculates for each p53+ cell the distance to the nearest CD3+ cell. A histogram can then be constructed. Preferably, increases in the proximity of the T cells among the cancer cells is increased by at least 2 fold (distance between cancer cell and nearest T cell is decreased by 2 fold), 3 fold (distance between cancer cell and nearest T cell is decreased by 3 fold), 4 fold (distance between cancer cell and nearest T cell is decreased by 4 fold) or 5 fold (distance between cancer cell and nearest T cell is decreased by 5 fold).

When effector CD3+ T-cells are in close proximity to the cancerous tumor cell, effector response ensues. Otherwise, an immunological barrier exists that allows tumor evasion mechanisms.

In the present invention, “frequency” is defined as the quantitative increase in T-cells and even more preferably effector CD3+ T-cells that are found among the cancer cells in the tumor microenvironment. Preferably, increases in frequency of the T cells among the cancer cells is increased by at least 20%, at least 30%, at least 40%, at least 50%, at least 60%, at least 70%, at least 80%, at least 90%, at least 100%, at least 200%, or at least 300%.

Examples of tumors include, but are not limited to, sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, esophageal cancer, pancreas cancer, renal cancer, stomach cancer, multiple myeloma and cerebral cancer. Preferred embodiments of tumors are adenocarcinomas. In some embodiments, the cancer may be pancreatic cancer, for example pancreatic ductal adenocarcinoma.

### B. T Cell Exclusion in Tumors

“T cell exclusion” in a tumor is defined as those tumor evasion mechanisms known in the art where effector CD3+ T cell subsets are prevented from being recruited to and accumulating among cancer cells within the tumor microenvironment. Tumor evasion mechanisms include, but are not limited to: (1) immunologic barriers within the tumor microenvironment, including a failure of immunosurveillance in the tumor, (2) non-functional antigen presenting cells, and (3) dysfunctional CD4+ T cells, CD8+ T cells, and excessive numbers of Foxp3+ regulatory T cells, A model of human PDA was developed to replicate a failure of immunosurveillance in the tumor. This failure is attributable to local immunosuppression mediated by the FAP+ stromal cell, which manifests as exclusion and likely death of T cells from regions of the tumor containing PDA cells and involves its production of CXCL12. As disclosed herein, the C—X—C motif receptor 4 (CXCR4) was found to mediate immune suppressive processes within the tumor microenvironment. Moreover, it was surprisingly found that inhibiting CXCR4, the CXCL12 receptor, promotes effector CD3+ T cell accumulation in cancer cell-containing regions of a tumor and inhibits tumor growth by eliminating the cancer cells.

In the prior art, expression of CXCL12 is associated with both impairment and promotion of immune control of growth of tumors. The art that demonstrates impairment of immune control (Righi et al.) indicates that this results from the recruitment of FoxP3+ regulatory T-cells to the tumor by expression of CXCL12. Surprisingly, it was observed that CXCL12 expression results in exclusion of all T cells, including CD4+ T cells, CD8+ T cells, and Foxp3+ regulatory T cells. The use of a CXCR4 signaling inhibitor removes this exclusion, and an increase in FoxP3+ cells as well as the other CD3+ T cell subsets at the tumor site was observed which is responsible for the elimination of PDA cells.

Furthermore, it is the interaction of CXCL12 coating the cancer cells with CXCR4 on infiltrating T cells that causes their apoptosis. This may be one mechanism of T cell exclusion from vicinity of cancer cells.

Accordingly, the present invention provides a method for recruitment of CD3+ T cell subsets, including CD4+ T cells, CD8+ T cells, and Foxp3+ regulatory T cells, to cancer cell-containing regions of a tumor in a subject, and methods for treating tumors by restoring immunological control of tumor growth. In this manner, the present invention overcomes the problem of T-cell exclusion and allows effector CD3+ T cell subsets to accumulate and recruit to the cancer cells in order to carry out their endogenous function of eliminating the cancer cells.

Therefore, the described method herein increases the recruitment of effector CD3+ T cell accumulation in the sites of a tumor that contain cancer cells, comprising administering to a subject in need thereof a pharmaceutically effective amount of an inhibitor of CXCR4 signaling.

The efficacy of the present invention is based on the observation that FAP+ stromal cells secrete CXCL12, which is a CXCR4 ligand. Administration of a CXCR4 signaling inhibitor as described herein, such as for example AMD3100, BMS-936564/MDX-1338, AMD11070, or LY2510924, results in inhibition of CXCR4 signaling and leads to a reduction in the observed immune suppression and removal of T cell exclusion. As a result, due to overcoming signaling by CXCR4 in response to the product of the FAP+ cells, CXCL12, CD3+ effector T-cells are recruited to the cancer cell-containing sites of the tumor and are able to eliminate the cancer cells.

For example, in one preferred embodiment of the present invention, AMD3100, BMS-936564/MDX-1338, AMD11070, or LY2510924, are examples of CXCR4 signaling inhibitor that can be used to recruit CD3+ T-cells to the cancer cell-containing sites of tumors and restore immunological regulation of the cancerous tumor cells. This restoration of immunological surveillance of the cancerous tumor results from removing T-cell exclusion and leading to the elimination of the cancerous cells.

In one preferred example, the described invention increases T-cell accumulation and recruitment to the cancerous tumor cells, such as PDA, to reduce the tumor growth and overcome tumor evasion mechanisms. For example, like most solid tumors, PDA tumors contain stromal cells that express fibroblast activation protein (FAP). FAP+ stromal cells are found in both PDA and other tumors and are known to secrete CXCL12, the chemokine that binds to CXCR4. One tumor evasion strategy is for cancer cells to bind CXCL12 and suppress local immune regulation of the tumor by excluding effector T cells from accumulating amongst the cancer cells. In the presence of a CXCR4 signaling inhibitor, such as, for example, AMD3100, BMS-936564/MDX-1338, AMD11070, or LY2510924, immune regulation of the tumor is restored.

Specifically, it is the recruitment of CD3+ T-cells that accumulate to the cancerous cells, such as PDA cells, when in the presence of a CXCR4 signaling inhibitor, such as, for example, AMD3100, BMS-936564/MDX-1338, AMD11070, or LY2510924, and these T-cells restore immunological regulation of the tumor. In addition, the CXCR4 signaling inhibitor increases T-cell accumulation at the cancer-cell containing sites of the tumor. The CXCR4 signaling inhibitor also reduces immune suppression in a cancerous tumor comprised of FAP+ stromal cells in an individual. Another function of the CXCR4 signaling inhibitor includes the infiltration of effector T-cells amongst the cancer cells. Such CD3+ T-cells include CD4+ T cells, CD8+ T cells, and Foxp3+ regulatory T cells.

As a result, this invention provides a method to treat a cancer comprised subject, such as a subject who contains PDA, by administering to a subject in need thereof a pharmaceutically effective amount of an inhibitor of CXCR4 signaling. Manufacture and medication of a CXCR4 signaling inhibitor is able to reduce immune suppression, increase infiltration of effector T-cells amongst the cancer cells, restore immunological regulation of the tumor, increase the sensitivity of the effector T-cells to the cancer cells, and effectively reduce and eliminate cancer cells, preferably, in a tumor comprised of FAP+ stromal cells.

This invention relates to the use of CXCR4 signaling inhibitors to reduce or abolish tumor immunosuppression in an individual with cancer. The CXCR4 signaling inhibitor described here can be used to increase the effectiveness of immune responses against cancer cells in a subject, in particular cell-mediated immune responses.

The CXCR4 signaling inhibitor as described herein reduces the ability of the cancerous tumor to suppress immune responses, for example by excluding CD3+ T cell subsets, such that immune responses to the tumor are more effective in the subject. This may have a beneficial therapeutic effect on the cancerous tumor of a human patient.

Provided in this description are methods of cancer immunotherapy in an individual in need thereof, which comprise administering to the individual a CXCR4 signaling inhibitor as described herein in an amount effective to treat the cancer, for example by increasing the effectiveness of the host immune response against the cancer in the individual.

Also provided herein are methods of reducing immune suppression in a cancerous tumor in an individual and/or increasing the effectiveness of an immune response, preferably a cell-mediated immune response, to a cancerous tumor in an individual, comprising administering a CXCR4 signaling inhibitor to the individual, as described herein.

Also provided herein are methods of increasing the sensitivity of a cancerous tumor in an individual to host immune responses, the method comprising administering a CXCR4 signaling inhibitor as described in the present invention to an individual.

Also provided herein are methods for increasing T cell accumulation and recruitment, preferably effector CD3+ T cell accumulation in the sites of a cancerous tumor that contain cancer cells, the method comprising administering a CXCR4 signaling inhibitor as described herein, such as, for example, AMD3100, BMS-936564/MDX-1338, AMD11070, or LY2510924. A CXCR4 signaling inhibitor can be used to increase T cell accumulation and recruitment at the cancer containing sites in a tumor. The present invention also relates to the use of a CXCR4 signaling inhibitor in the manufacture of a medication or use in increasing T cell accumulation at the cancer containing sites in a tumor.

We have observed that, before treatment, most T cells are found in the stromal regions of the tumor. This distribution of T-cells is believed to be at least partially responsible for the inability of the immune response to the cancer cells to control tumor growth. The administration of a CXCR4 signaling inhibitor increases the accumulation of effector T-cells at in the cancer cell regions of the tumor.

Tumor therapy, as referred to herein, includes therapies which reduce the rate of tumor growth, that is slow down, but do not necessarily eliminate tumor growth.

Reduction in the rate of tumor growth can be, for example, a reduction in at least 10%, 20%, 30%, 40%, 50%, 75%, 100%, 150%, 200% or more of the rate of growth of a tumor. For example, the rate of growth can be measured over 1, 2, 3, 4, 5, 6 or 7 days, or for longer periods of one or more weeks.

In some embodiments, the invention may result in the arrest of tumor growth, or the reduction in tumor size or the elimination of a tumor.

Cancer cells within the tumor in the subject may be immunologically distinct from normal somatic cells in the subject (for example, the tumor may be immunogenic; alternatively, even if it is not immunogenic, it may present different immunological determinants(s) from somatic cells). For example, the cancer cells may be capable of eliciting a systemic immune response in the subject against one or more antigens expressed by the cancer cells. The antigens that elicit the immune response may be tumor antigens or may be shared by normal cells.

In embodiments, the tumor, although presenting different antigenic determinants, is hidden from the immune system of a subject and displays tumor evasion characteristics. For example, the tumor may exclude immune cells, thus lowering its immunological visibility and sensitivity, and/or preventing the immune system from acting to attack the tumor.

CD8+ T cells that are specific for cancer cells within the cancerous tumor may be present in the subject.

In embodiments, CD8+ T cells are absent from the cancerous tumor or are absent from regions of the tumor that contain cancer cells within a critical distance required for activation by antigens expressed by the cancer cells. In some embodiments, the cancer cells may express one or more antigens that are not expressed by normal somatic cells in the subject (i.e. tumor antigens). Tumor antigens are known in the art and may elicit immune responses in the subject. In particular, tumor antigens may elicit T cell-mediated immune responses against cancer cells in the subject i.e. the tumor antigens may be recognized by CD8+ T cells in the subject.

Tumor antigens expressed by cancer cells in a cancerous tumor may include, for example, cancer-testis (CT) antigens encoded by cancer-germ line genes, such as MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2(HOM-MEL-40), SSX-3, SSX-4, SSX-5, SCP-I and XAGE and immunogenic fragments thereof (Simpson et al., Nature Rev (2005) 5, 615-625, Gure et al., Clin Cancer Res (2005) 11, 8055-8062; Velazquez et al., Cancer Immun (2007) 7, 11; Andrade et al., Cancer Immun (2008) 8, 2; Tinguely et al., Cancer Science (2008); Napoletano et al., Am J of Obstet Gyn (2008) 198, 99 e91-97).

Other tumor antigens that may be expressed include, for example, overexpressed or mutated proteins and differentiation antigens particularly melanocyte differentiation antigens such as p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR.alpha. fusion protein, PTPRK, K-ras, N-ras, Triosephosphate isomeras, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\ cyclophilin C-associated protein), TAAL6, TAG72, TLP, and TPS and tyrosinase related proteins such as TRP-1, TRP-2, and mesothelin.

Other tumor antigens that may be expressed include out-of-frame peptide-MHC complexes generated by the non-AUG translation initiation mechanisms employed by “stressed” cancer cells (Malarkannan et al. Immunity 1999).

Other tumor antigens that may be expressed are well-known in the art (see for example WO00/20581; Cancer Vaccines and Immunotherapy (2000) Eds Stern, Beverley and Carroll, Cambridge University Press, Cambridge) The sequences of these tumor antigens are readily available from public databases but are also found in WO 1992/020356 A1, WO 1994/005304 A1, WO 1994/023031 A1, WO 1995/020974 A1, WO 1995/023874 A1 & WO 1996/026214 A1.

In some embodiments, a cancerous tumor suitable for treatment as described herein may be resistant to immunotherapy in the absence of a CXCR4 signaling inhibitor. For example, the cancer cells within a cancerous tumor may express PD-L1. PD-L1 expression may be spontaneous in the cancer cells or may occur as a result of the inhibition of CXCR4 signaling. CXCR4 signaling inhibition allows T cells to infiltrate the cancer regions of the tumor and secrete IFN-gamma, which induces PD-L1 expression by epithelial cells, including epithelial cancer cells.

A subject suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

In some embodiments, the subject is a human. In other embodiments, non-human mammals, especially mammals that are conventionally used as models for demonstrating therapeutic efficacy in humans (e.g. murine, primate, porcine, canine, or rabbit animals) may be employed.

In some embodiments, the subject may have minimal residual disease (MRD) after an initial cancer treatment.

A subject with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in a subject may include identification of a particular cell type (e.g. a cancer cell) in a sample of a body fluid or tissue obtained from the subject.

Inhibition of CXCR4 signaling in a cancerous tumor may increase the accumulation of T cells into regions of the cancerous tumor that contain cancer cells.

Preferred CXCR4 signaling inhibitors may reduce or abolish CXC12-mediated CXCR4 signaling activity to the same or greater extent than AMD3100 (Plerixafor) under the same conditions. For example, a CXCR4 signaling inhibitor may have a potency that is equal to or greater than the potency of AMD3100 (e.g. an IC50 of about 650 nM or less; Fricker et al Biochem Pharmacol 72 (5) 588-596). In other preferred embodiments, the CXCR4 signaling inhibitor described herein is at least 10%, 20%, 30%, 40%, 50%, 75%, 100%, 150%, 200% or 300% as potent as AMD3100.

### C. Formulations

A suitable serum concentration of CXCR4 signaling inhibitor for the effective blockage of the binding of CXCL12 by CXCR4 may be readily determined from the affinity of the inhibitor for CXCR4 or CXCL12.

The CXCR4 signaling inhibitor may be administered together with other anti-cancer therapies, such as conventional chemotherapeutic agents, radiation therapy or cancer immunotherapy. For example, the CXCR4 signaling inhibitor is administered together with an anti-cancer compound. The CXCR4 signaling inhibitor and the anti-cancer compound may be separate compounds or molecules or they may be covalently or non-covalently linked in a single compound, molecule, particle or complex.

An anti-cancer compound may be any anti-cancer drug or medicament which has activity against cancer cells. Suitable anti-cancer compounds for use in combination with CXCR4 as disclosed herein may include aspirin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGF/VEGF receptors, platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; and anti-virals such as oseltamivir phosphate, Amphotericin B, and palivizumab.

While it is possible for CXCR4 signaling inhibitors and anti-cancer compounds to be administered alone, it is preferable to present the compounds in the same or separate pharmaceutical compositions (e.g. formulations).

A pharmaceutical composition may comprise, in addition to the CXCR4 signaling inhibitor and/or an anti-cancer compound, one or more pharmaceutically acceptable carriers, adjuvants, excipients, diluents, fillers, buffers, stabilizers, preservatives, lubricants, or other materials well known to those skilled in the art. Suitable materials will be sterile and pyrogen-free, with a suitable isotonicity and stability. Examples include sterile saline (e.g. 0.9% NaCl), water, dextrose, glycerol, ethanol or the like or combinations thereof. Such materials should be non-toxic and should not interfere with the efficacy of the active compound. The precise nature of the carrier or other material will depend on the route of administration, which may be by bolus, infusion, injection or any other suitable route, as discussed below. Suitable materials will be sterile and pyrogen free, with a suitable isotonicity and stability. Examples include sterile saline (e.g. 0.9% NaCl), water, dextrose, glycerol, ethanol or the like or combinations thereof. The composition may further contain auxiliary substances such as wetting agents, emulsifying agents, pH buffering agents or the like.

Suitable carriers, excipients, etc. can be found in standard pharmaceutical texts, for example, Remington's Pharmaceutical Sciences, 18th edition, Mack Publishing Company, Easton, Pa., 1990.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

In some embodiments, one or both of the CXCR4 signaling inhibitor may be provided in a lyophilized form for reconstitution prior to administration. For example, lyophilized reagents may be re-constituted in sterile water and mixed with saline prior to administration to a subject

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Optionally, other therapeutic or prophylactic agents may be included in a pharmaceutical composition or formulation.

Reducing immune suppression in tumors as described herein may be useful in immunotherapy for the treatment of cancer.

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment as a prophylactic measure (i.e. prophylaxis) is also included. For example, a subject susceptible to or at risk of the occurrence or re-occurrence of cancer may be treated as described herein. Such treatment may prevent or delay the occurrence or re-occurrence of cancer in the subject.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic T-lymphocytes, and a decrease in levels of tumor-specific antigens. Reducing immune suppression in cancerous tumors in a subject may improve the capacity of the subject to resist cancer growth, in particular growth of a cancer already present the subject and/or decrease the propensity for cancer growth in the subject.

CXCR4 signaling inhibitors may be administered as described herein in therapeutically-effective amounts.

The term “therapeutically-effective amount” as used herein, pertains to that amount of an active compound, or a combination, material, composition or dosage form comprising an active compound, which is effective for producing some desired therapeutic effect, commensurate with a reasonable benefit/risk ratio.

It will be appreciated that appropriate dosages of the active compounds can vary from patient to patient. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the active compound, other drugs, compounds, and/or materials used in combination, and the age, sex, weight, condition, general health, and prior medical history of the patient. The amount of active compounds and route of administration will ultimately be at the discretion of the physician, although generally the dosage will be to achieve concentrations of the active compound at a site of therapy without causing substantial harmful or deleterious side-effects.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

For example, a CXCR4 signaling inhibitor as described herein, such as such as, for example, AMD3100, BMS-936564/MDX-1338, AMD11070, or LY2510924 may be administered by continuous intravenous infusion in an amount sufficient to maintain the serum concentration at a level that yields >90% inhibition of CXCL12 binding by CXCR4 (see for example Hendrix et al J Acquir Immune Defic Syndr. 2004 Oct. 1; 37(2):1253-62). Other CXCR4 signal inhibitors described herein can also be used in this same manner.

Administration in vivo can be effected in one dose, continuously or intermittently (e.g., in divided doses at appropriate intervals). Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the physician.

Administration of anti-cancer compounds and the CXCR4 signaling inhibitor may be simultaneous, separate or sequential. By “simultaneous” administration, it is meant that the anti-cancer compounds and the CXCR4 signaling inhibitor are administered to the subject in a single dose by the same route of administration.

By “separate” administration, it is meant that the anti-cancer compounds and the CXCR4 signaling inhibitor are administered to the subject by two different routes of administration which occur at the same time. This may occur for example where one agent is administered by infusion or parenterally and the other is given orally during the course of the infusion or parenteral administration.

By “sequential” it is meant that the anti-cancer compounds and the CXCR4 signaling inhibitor are administered at different points in time, provided that the activity of the first administered agent is present and ongoing in the subject at the time the second agent is administered. For example, the anti-cancer compounds may be administered first, such that an immune response against a tumor antigen is generated, followed by administration of the CXCR4 signaling inhibitor, such that immunosuppression at the site of the tumor is reduced, or vice versa. Preferably, a sequential dose will occur such that the second of the two agents is administered within 48 hours, preferably within 24 hours, such as within 12, 6, 4, 2 or 1 hour(s) of the first agent.

Multiple doses of the CXCR4 signaling inhibitor may be administered, for example 2, 3, 4, 5 or more than 5 doses may be administered after administration of the anti-cancer compounds. The administration of the CXCR4 signaling inhibitor may continue for sustained periods of time after administration of the anti-cancer compounds. For example treatment with the CXCR4 signaling inhibitor may be continued for at least 1 week, at least 2 weeks, at least 3 weeks, at least 1 month or at least 2 months. Treatment with the CXCR4 signaling inhibitor may be continued for as long as is necessary to achieve complete tumor rejection.

Multiple doses of the anti-cancer compounds may be administered, for example 2, 3, 4, 5 or more than 5 doses may be administered after administration of the CXCR4 signaling inhibitor. The administration of the anti-cancer compounds may continue for sustained periods of time after administration of the CXCR4 signaling inhibitor. For example treatment with the anti-cancer compounds may be continued for at least 1 week, at least 2 weeks, at least 3 weeks, at least 1 month or at least 2 months. Treatment with the anti-cancer compounds may be continued for as long as is necessary to achieve complete tumor rejection.

The active compounds or pharmaceutical compositions comprising the active compounds may be administered to a subject by any convenient route of administration, whether systemically/peripherally or at the site of desired action, including but not limited to, oral (e.g. by ingestion); and parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Usually administration will be by the intravenous route, although other routes such as intraperitoneal, subcutaneous, transdermal, oral, nasal, intramuscular or other convenient routes are not excluded.

The pharmaceutical compositions comprising the active compounds may be formulated in suitable dosage unit formulations appropriate for the intended route of administration.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Compositions comprising anti-cancer compounds and/or CXCR4 signaling inhibitors may be prepared in the form of a concentrate for subsequent dilution, or may be in the form of divided doses ready for administration. Alternatively, the reagents may be provided separately within a kit, for mixing prior to administration to a human or animal subject.

The CXCR4 signalling inhibitor may be administered alone or in combination with other treatments, either simultaneously or sequentially dependent upon the individual circumstances. For example, CXCR4 signalling inhibitors as described herein may be administered in combination with one or more additional active compounds.

In some embodiments, the treatment of a subject using a CXCR4 signaling inhibitor as described herein may further comprise administering one or more immunotherapeutic agents to the subject.

An immunotherapeutic agent may facilitate or enhance the targeting of cancer cells by the immune system, in particular T cells, through the recognition of antigens expressed by the cancer cells.

Suitable agents include adoptive T cell therapies and cancer vaccine preparations designed to induce T lymphocytes (T cells) recognizing a localized region of an antigen or epitope specific to the tumor cell.

A cancer vaccine is an agent, a cell-based agent, molecule, or immunogen which stimulates or elicits an endogenous immune response in a subject or subject against one or more tumor antigens. Suitable cancer vaccines are known in the art and may be produced by any convenient technique.

The use of tumor antigens to generate immune responses is well-established in the art (see for example; Kakimi K, et al. Int J Cancer. 2011 Feb. 3; Kawada J, Int J Cancer. 2011 Mar. 16; Gnjatic S, et al. Clin Cancer Res. 2009 Mar. 15; 15(6):2130-9; Yuan J, et al. Proc Natl Acad Sci USA. 2008 Dec. 23; 105(51):20410-5; Sharma P, et al. J Immunother. 2008 November-December;31(9):849-57; Wada H, et al. Int J Cancer. 2008 Nov. 15; 123(10):2362-9; Diefenbach C S, et al. Clin Cancer Res. 2008 May 1; 14(9):2740-8; Bender A, et al. Cancer Immun. 2007 Oct. 19; 7:16; Odunsi K, et al. Proc Natl Acad Sci USA. 2007 Jul. 31; 104(31):12837-42; Valmori D, et al. Proc Natl Acad Sci USA. 2007 May 22; 104(21):8947-52; Uenaka A, et al. Cancer Immun. 2007 Apr. 19; 7:9; Kawabata R, et al. Int J Cancer. 2007 May 15; 120(10):2178-84; Jäger E, et al. Proc Natl Acad Sci USA. 2006 Sep. 26; 103(39):14453-8; Davis ID Proc Natl Acad Sci USA. 2005 Jul. 5; 102(27):9734; Chen Q, Proc Natl Acad Sci USA. 2004 Jun. 22; 101(25):9363-8; Jäger E, Proc Natl Acad Sci USA. 2000 Oct. 24; 97(22):12198-203; Carrasco J, et al. J Immunol. 2008 Mar. 1; 180(5):3585-93; van Baren N, et al. J Clin Oncol. 2005 Dec. 10; 23(35):9008-21; Kruit W H, et al. Int J Cancer. 2005 Nov. 20; 117(4):596-604; Marchand M, et al. Eur J Cancer. 2003 January; 39(1):70-7; Marchand M et al. Int J Cancer. 1999 Jan. 18; 80(2):219-30; Atanackovic D, et al. Proc Natl Acad Sci USA. 2008 Feb. 5; 105(5):1650-5).

Cancer cells from the subject may be analyzed to identify a tumor antigen expressed by the cancer cells. For example, a method as described herein may comprise the step of identifying a tumor antigen which is displayed by one or more cancer cells in a sample obtained from the subject. A cancer vaccine comprising one or more epitopes of the identified tumor antigen may then be administered to the subject whose cancer cells express the antigen. The vaccine may induce or increase an immune response, preferably a T cell mediated immune response, in the subject against the cancer cells expressing the identified tumor antigen.

The cancer vaccine may be administered before, at the same time, or after the CXCR4 signaling inhibitors are administered to the subject as described here.

Adoptive T cell therapy involves the administration to a subject of tumor-specific T cells to a subject. Preferably, the T cells were previously isolated from the subject and expanded ex vivo. Suitable adoptive T cell therapies are well known in the art (J. Clin Invest. 2007 Jun. 1; 117(6): 1466-1476.) For example, adoptive T cell therapy using CAR T cells (chimeric antigen receptor) would be greatly improved if used in combination with a CXCR4 signaling inhibitor. CAR T cells must migrate into a tumor to get in proximity to the cancer cells within the tumor in order to mediate their killing activity. The present invention, such as such as, for example, AMD3100, BMS-936564/MDX-1338, AMD11070, or LY2510924, used in combination with CAR T cells may improve this type of immunotherapy.

In some embodiments, the treatment of an individual using a CXCR4 signalling inhibitor may further comprise administering one or more tumor therapies to treat the cancerous tumor. Such therapies include, for example, tumor medicaments, radiation and surgical procedures.

A tumor medicament is an agent which is administered to a subject for the purpose of treating a cancer. Suitable medicaments for the treatment of tumors are well known in the art.

Suitable medicaments for use in combination with CXCR4 signalling inhibitors as disclosed herein may include aspirin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumour necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGF/VEGF receptors, platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; and anti-virals such as oseltamivir phosphate, Amphotericin B, and palivizumab.

Additionally, other T cell checkpoint antagonists, like Lag-3, or inhibitors of IDO1/IDO2 (indoleamine 2,3-dioxygenase) could also be used in combination with the present invention. These enzymes catabolize tryptophan in the tumor microenvironment, which impairs T cell function. By using a CXCR4 signaling inhibitor, such as for example, AMD3100, BMS-936564/MDX-1338, AMD11070, or LY2510924, in combination with a T cell checkpoint antagonist may synergistically increase cancer cell killing within a tumor.

Various embodiments are disclosed above for a CXCR4 signalling inhibitor. Aspects and embodiments of the invention relating to a CXCR4 signalling inhibitor and optionally one or more other agents disclosed above include disclosure of the administration of the compounds or agents separately (sequentially or simultaneously) or in combination (co-formulated or mixed). For each aspect or embodiment, the specification further discloses a composition comprising the CXCR4 signalling inhibitor and optionally one or more other agents co-formulated or in admixture with each other and further discloses a kit or unit dose containing the CXCR4 signalling inhibitor e. Optionally, such compositions, kits or doses further comprise one or more carriers in admixture with the agent or co-packaged for formulation prior to administration to an individual.

Immunosuppression is shown herein to result from the exclusion of T cells from the microenvironment of the cancerous tumor. Inhibition of CXCR4 signalling using a CXCR4 signalling inhibitor, such as AMD3100, as described herein, overcomes this exclusion and exposes cancer cells in the tumor to T cells. In other aspects of the invention, methods of treatment may comprise the administration of a CXCR4 signalling inhibitor in combination with an immunotherapeutic agent, as described above, such as a cancer vaccine or adoptive T cell therapy, for the treatment of cancer. The CXCR4 signalling inhibitor and immunotherapeutic agent may be administered in the absence of a PD-1 signalling inhibitor.

Suitable CXCR4 signalling inhibitors, immunotherapeutic agents and methods of treatment are described mutatis mutandis above.

Various embodiments are also disclosed above for combinations of a PD-1 signaling inhibitor and a CXCR4 signaling inhibitor. Aspects and embodiments of the invention relating to combinations of a PD-1 signaling inhibitor and a CXCR4 signaling inhibitor and optionally one or more other agents disclosed above include disclosure of the administration of the compounds or agents separately (sequentially or simultaneously) or in combination (co-formulated or mixed). For each aspect or embodiment, the specification further discloses a composition comprising the PD-1 signaling inhibitor and CXCR4 signaling inhibitor and optionally one or more other agents co-formulated or in admixture with each other and further discloses a kit or unit dose containing the PD-1 signaling inhibitor and CXCR4 signaling inhibitor packaged together, but not in admixture. Optionally, such compositions, kits or doses further comprise one or more carriers in admixture with one or both agents or co-packaged for formulation prior to administration to a subject.

Immunosuppression is shown herein to result from the exclusion and/or death of T cells from the microenvironment of the cancerous tumor Inhibition of CXCR4 signaling using a CXCR4 signaling inhibitor, such as, for example, AMD3100, BMS-936564/MDX-1338, AMD11070, or LY2510924, as described herein, overcomes this exclusion and exposes cancer cells in the tumor to T cells. In other aspects of the invention, methods of treatment may comprise the administration of a CXCR4 signaling inhibitor in combination with an immunotherapeutic agent, as described above, such as a cancer vaccine or adoptive T cell therapy, for the treatment of cancer. The CXCR4 signaling inhibitor and immunotherapeutic agent may be administered in the absence of the PD-1 signaling inhibitor.

Suitable CXCR4 signaling inhibitors, immunotherapeutic agents and methods of treatment are described mutatis mutandis above.

Various further aspects and embodiments of the present invention will be apparent to those skilled in the art in view of the present disclosure.

Other aspects and embodiments of the invention provide the aspects and embodiments described above with the term “comprising” replaced by the term “consisting of” and the aspects and embodiments described above with the term “comprising” replaced by the term “consisting essentially of”.

“and/or” where used herein is to be taken as specific disclosure of each of the two specified features or components with or without the other. For example “A and/or B” is to be taken as specific disclosure of each (i) A, (ii) B and (iii) A and B, just as if each is set out individually.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such these are within the scope of the present invention.

All documents and sequence database entries mentioned in this specification are incorporated herein by reference in their entirety for all purposes.

The invention is further described below, with reference to the following examples.

### EXAMPLES

### Methods

### Mice

All experiments were performed in accordance with institutional guidelines and were approved by the UK Home Office and the animal ethics committee of CRUK and the University of Cambridge. Mice were housed at a 12-hour light/12-hour dark cycle and received diet and water ad libitum. The generation of LSL-KrasG12D/+;LSL10 Tp53R172H/+;Pdx-1-Cre (KPC) and FAP-DTR BAC transgenic mice has been described previously (8, 10). These strains were crossed to generate KPCD (Kras;p53;Cre;DTR) mice. KPC and KPCD mice were screened for tumors from an age of 60 days by abdominal palpation. Tumors were verified by high-resolution ultrasound (Vevo 2100, VisualSonics). Mice with average tumor diameters between 5-8 mm (corresponding to approx. 200 mm3 volume) were enrolled on 6 day treatment studies with 2 follow-up tumor size measurements (day 3 and 6). Where possible, tumors were assessed at multiple angles and the volumes averaged. KPC(−/+DTR Tg) mice were treated every 48 h with 25 ng/g DTx (List Biologicals) in PBS, 160 μg α-PD1 (10F.9G2, Biolegend), 100 μg α-CTLA-4 (9H10, Biolegend) or isotype control antibody by intraperitoneal injection. AMD3100 (SigmaAldrich) was administered by osmotic pump (inserted on day 0) at 30 mg/ml or 90 mg/ml (high dose). For T-cell depletion studies mice received 300 μg each of α-CD4 (GK1.5, Biolegend) and α-CD8α (53-6.7, Bioloegend) or respective isotype control antibodies for 3 consecutive 24 days before treatment start and on days 2 and 5 during the course of treatment via intraperitoneal injection.

Subcutaneous LL2/OVA Tumor Model:

C57BL/6 were purchased from Charles River UK and Rag2−/− mice were bred at the local establishment. 2×105 LL2/OVA cells were injected subcutaneously in RPMI with 1% heat inactivated mouse serum. Tumor sizes were measured using calipers, measuring the long (L) and short (S) dimension, and tumor volumes were calculated using the equation: volume=(L×S2)/2. AMD3100 (30 mg/ml) treatment commenced on day 12 when tumors reached at least 62 mm3 by inserting ALZET osmotic pumps (1007D or 2002, Charles River) subcutaneously.

### Cell Lines

The generation of Lewis lung carcinoma cell line LL2 expressing chicken ovalbumin (LL2/OVA) was reported in Kraman et al 2010. The pancreatic cancer cell lines K8484 and TB32964 were derived from tumors arising in KPC mice. They were cultured in DMEM supplemented with 10% FCS.

### ELISpot Assays

Single cell suspensions of whole tumors were stained with α-CD3-PE (clone 17A2, eBioscience) to allow MACS depletion of T cells using αPE magnetic beads (Miltenyi Biotech). CD8+ T cells were isolated from whole spleen using the untouched CD8α+ T cell Isolation Kit II (Miltenyi Biotech) according to the manufacturer's instructions. Purity was confirmed by flow cytometry. Doubling dilutions of CD8+ T cells from KPC, KC and PC mice were challenged with a constant number of stimulator cells (freshly isolated tumor cells from KPC tumor-bearing mice; tumor cell lines established from KPC mice; and freshly isolated PanIN cell from pre-tumor bearing KPC mice) in a 12 hour IFN-γ release ELISpot assay according to manufacturer's instructions (BD Biosciences). Plates were read using an AID ELISpot Plate Reader v3.5 (Autoimmun Diagnostika). The frequency of IFN-γ secreting CD8+ T cells was calculated from a dose-response curve.

### Immunofluorescence (IF)

5 μm frozen tissue sections were fixed in 4% paraformaldehyde (PFA) for 10 minutes at room temperature. Slides were blocked for one hour in 10% donkey serum (Sigma Aldrich)/0.2% Triton x-100. Primary antibodies were incubated overnight at 4° C. Following washing, slides were incubated for one hour at room temperature with appropriate secondary antibody and DAPI counterstain. Slides were subsequently incubated in 0.3M glycine for 10 minutes to reduce autofluorescence and mounted in Hydromount aqueous mounting medium (Fisher Scientific). Images were acquired on a Leica SP5 tandem confocal microscope. For analysis of p53 and Treg staining slides were scanned and analyzed using the automated ARIOL XT system.

### Immunohistochemistry (IHC)

Archival paraffin sections from the University of Cambridge Addenbrooke's Hospital tissue bank were used in accordance with institutional and national policies.

Immunohistochemical assessment of FAP, 1 p53, CXCL12 and CD3 was performed. 3 μm formalin-fixed, paraffin-embedded tissue sections were deparaffinised, rehydrated in an ethanol series, antigen-retrieved in 0.01M citrate buffer (pH6)/Proteinase K, and endogenous peroxidase quenched with 3% H2O2. Sections were blocked in 1% normal donkey serum and Avidin/Biotin Blocking Kit (Vector Laboratories), and incubated consecutively with primary antibody or rabbit/sheep Immunoglobulin (Vector Labs), biotinylated secondary antibody (Jackson

ImmunoResearch Labs), and Vectastain ABC Reagent (Vector Labs). Immunopositive cells were visualized by liquid DAB-substrate-chromogen system (DAKO).

P53 and CD3 stainings were carried out on the BondMax Autostainer (Vision Biosystems). Briefly, antigen retrieval was performed at 100° C. in Bond Citrate buffer, followed by 15 min incubation with primary antibody at room temperature, 8 min postprimary step, 8 min incubation with polymer (Bond Polymer Detection System; Vision Biosystems), and colorimetric development with diaminobenzidine (Vision Bio-systems). Slides were counterstained with haematoxylin and imaged on the ARIOL XT system.

### Antibodies for IF and IHC

| Antigen | Clone/Cat.nr. | Supplier |
|---|---|---|
| CD11b | M1/70 | eBioscience |
| CD3 (human) | SP7 | Neomarkers |
| CD3 (mouse) | 17A2 | R & D Systems |
| CK19 | TROMA III | DSHB |
| CXCl12 (human) | Rabbit polyclonal | Peprotech |
| CXCL12 (mouse) | MAB350 | R & D Systems |
| FAP | Sheep polyclonal | R & D Systems |
| FoxP3 | FJK-16S | eBioscience |
| Ki67 | B56 | BD Biosciences |
| p53 (human) | D07 | Dako |
| p53 (mouse) | CM5 | Vector Labs |
| CD45 | AF114 | R & D Systems |
| SMA | ab5694 | Abcam |
| CD34 | RAM34 | eBioscience |

### Flow Cytometry

To prepare single cell suspensions, tissues were finely minced in 3 mg/ml Dispase II (Roche), 1 mg/ml Collagenase (Sigma), 1 mg/ml DNAse I (Roche) in RPMI and incubated for 1 hour at 37° C. with mechanical disruption using a pipette every 15 minutes. Following digestion, EDTA was added to a final 1 concentration of 10 mM for 5 minutes and cell suspensions passed through a 70 μm cell strainer. Antibody Fc receptor binding was blocked in 1% Fc blocking antibody (clone 2.4G2, BD Pharmingen) for 45 minutes on ice. For FAP staining, cells were incubated with sheep anti-FAP antibody (R&D Systems) at 10 μg/ml or sheep IgG control for 30 minutes on ice. Cells were subsequently washed, re-blocked and incubated with PE-conjugated donkey anti-sheep IgG secondary antibody (R&D systems) for 30 minutes, along with any directly conjugated primary antibodies. For analysis of viability cells were resuspended in 7AAD (Calbiochem). Data were collected on the LSRII flow cytometer (BD Bioscience) and analyzed using Flowjo software. Cell sorting was carried out using the BD FACSAria cell sorter.

| Antigen/Clone | Supplier | Concentration |
|---|---|---|
| FAP (cat. AF3715) | R&D systems | 10 μg/ml |
| CD45/30-F11 | eBioscience | 2 μg/ml |
| CD34/RAM-34 | eBioscience | 2 μg/ml |
| PDGFRa/APA5 | eBioscience | 2 μg/ml |
| CD11b/M1/70 | eBioscience | 2 μg/ml |
| CD31/390 | eBioscience | 5 μg/ml |

### RNA Analysis

RNA was extracted from RNAlater (Life Technologies) stabilized whole tumor samples following the RNeasy Plus mini kit protocol (QIAGEN) and using QIAGEN's tissue lyser for homogenization. 2 μg of RNA was reverse transcribed with Applied Biosystem's high capacity RNA to cDNA kit followed by real time PCR using Taqman primers on the 7900HT qPCR system (Fapa; Mm00484254_m1, Tbp: Mm00446971_m1). Delta Cts were calculated in relationship to Tbp endogenous control and further normalized to the mean induction over Tbp of the control group. For RNA analysis of sorted cell populations tumors were dissociated as for flow cytometric analysis and stained at 4° C. in 2% FCS/2 mM EDTA/PBS. Following red blood cell lysis, viable cells were sorted by a BD Influx Cell Sorter (BD Bioscience) into the following fractions: FAP+; CD11b+ for myeloid cells; and CD45−FAP−CD31− for PanIN/PDA cells. Total RNA was extracted from frozen cell pellets with the RNeasy Mini Kit (Qiagen) and RT-PCR was performed with TaqMan RNA-to-Ct 1-Step Kit (Life Technologies) on the ABI 7900HT Fast Real-Time PCR System (Applied Biosystems). The following Taqman Gene Expression Assays (Life Technologies) were used: Tbp Mm00446973_m 1 1; Cxcl12 Mm00445553_m1; Cxcr4 Mm01292123 ml. Data were normalised to Tbp.

### RNA-Seq and Computational Methods

RNA extraction and sequencing was performed as previously described (Roberts et al.). The short-read RNA-seq data generated in this investigation, along with T-helper cell RNA-seq data (Wei et al., 2011) (GEO accession GSE20898), and RNA-seq data for FAP+ cells and MEFs (Roberts et al., GEO accession GSE39438) were mapped using the Bowtie2 (Langmead and Salzberg, 2012) and aligned to the mouse mm9 reference genome. Subsequently, Tophat2 was used to map junction reads using the command-line switches “--GTF (gtffile) --b2-very-sensitive --b2-D 500 --b2-R 500 --solexa1.3-quals.” To calculate expression levels, Cufflinks2 (Trapnell et al., 2010) was used to calculate fragment per kilobase million values (FPKM) (“--output-dir $outpath --GTF $gtffile - p 8 --multi-read-correct --frag-bias-correct.”), and htseq version 0.5.3p4 to calculate kilobase million (RPKM) (“--quiet --stranded=no -a 30.”) values. The RNA-seq data generated in this investigation was deposited in the NCBI Gene Expression Omnibus (GEO) and can be accessed using the GEO accession number (GSE42605). Principle component analysis (PCA) was performed using the prcomp and predict functions in R v2.15.0.

### Statistical Analyses

Statistical analyses were carried out using GraphPad Prism version 6.0b for Mac OS X. For multiple comparisons ANOVA with Bonferroni's post hoc test was applied. In all other cases significance was determined using Student's t-test unless specified otherwise in the figure legend. Data are presented as mean−/+SEM. Statistical comparison of growth curves was performed using a permutation-based, pairwise test.

### In Vitro Assay

This assay tests the mechanism of T cell exclusion from the vicinity of the cancer cells. It also allows screening of drugs and tumors for potential efficacy. This in vitro assay may also be used to screen other drugs (anti-CXCL12, anti-CXCR4, other CXCR4-directed small molecules) that may interfere with the CXCL12/CXCR4 interaction.

300 um vibratome sections of fresh autochthonous mouse pancreatic tumors (the mouse KPC model of Hingorani et al., Cancer Cell 2005) are overlaid with labeled mouse splenic T cells that have been activated in vitro for several days with anti-CD3 and IL-2 (this generates a T cell population that is like the effector T cells that normally infiltrate inflamed tissues/tumors). The tumor tissue+labeled T cells are then cultured at 37° C. for up to 120 min, after which the non-infiltrated T cells are washed off. The vibratome sections are then fixed for staining (p53 which identifies cancer cells) and SHG (to visualize collagen/stromal regions) by confocal microscopy.

In control culture media, T cells localize to the stromal regions and are absent from regions containing cancer cells. This is the distribution of T cells that we published in the Dec PNAS paper. Culture in the presence of AMD3100 causes T cells to be found in both stromal and cancer cell regions, as occurs in vivo. Culture of vibratome sections with T cells in which CXCR4 has been deleted by CRISPR-Cas9 technology leads to T cells accumulating also amongst cancer cells. Culture in the presence of Z-VAD, a pan-caspase inhibitor, leads to T cells accumulating also amongst cancer cells.

### Example 1

### FAP+ Cells are Responsible for Immune Suppression

The mesenchymal tumoral stromal cell that is identified by its expression of the membrane protein, FAP, was shown recently to mediate immunosuppression in a transplanted tumor model (8). As FAP+ stromal cells are present in human PDA (9), we investigated whether this immunosuppressive activity of the murine FAP+ stromal cell might be involved in the resistance of this cancer to immunotherapy. In the present study, we demonstrate that the autochthonous KPC (LSL-KrasG12D/+; LSL-Trp53R172H/+;Pdx-1-Cre) model of PDA (10) replicates the resistance of human PDA to checkpoint antagonists, despite the presence of systemic anti-PDA immunity. This failure of immunosurveillance is attributable to local immunosuppression mediated by the FAP+ stromal cell.

In the KPC model, Cre-mediated expression of Trp53R172H and KrasG12D is targeted to the pancreas, causing the development of invasive and metastatic carcinoma that recapitulates many aspects of human PDA, including the loss of heterozygosity (LOH) of Trp53 (10). KPC mice with appropriately sized tumors demonstrate consistent tumor growth, which permits robust analyses of experimental interventions.

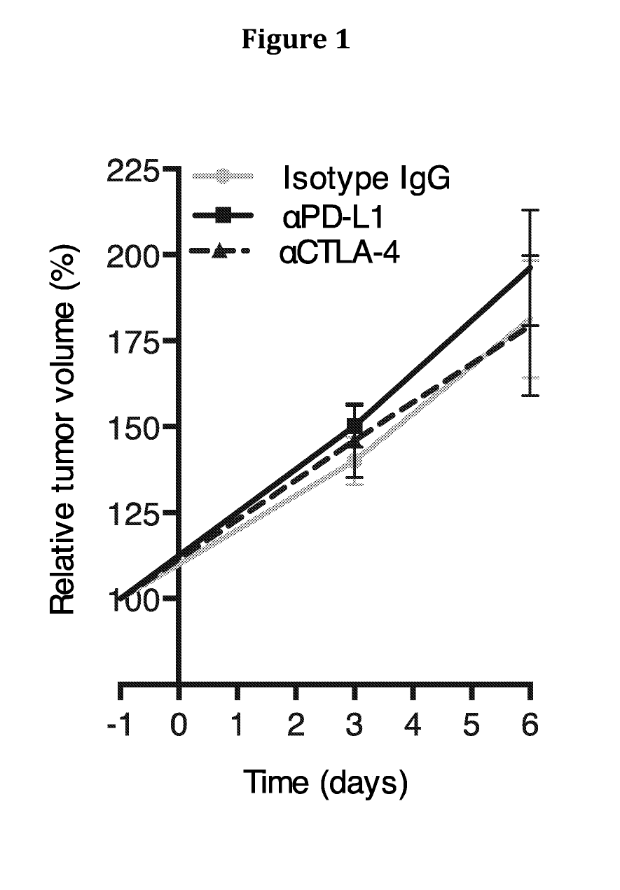

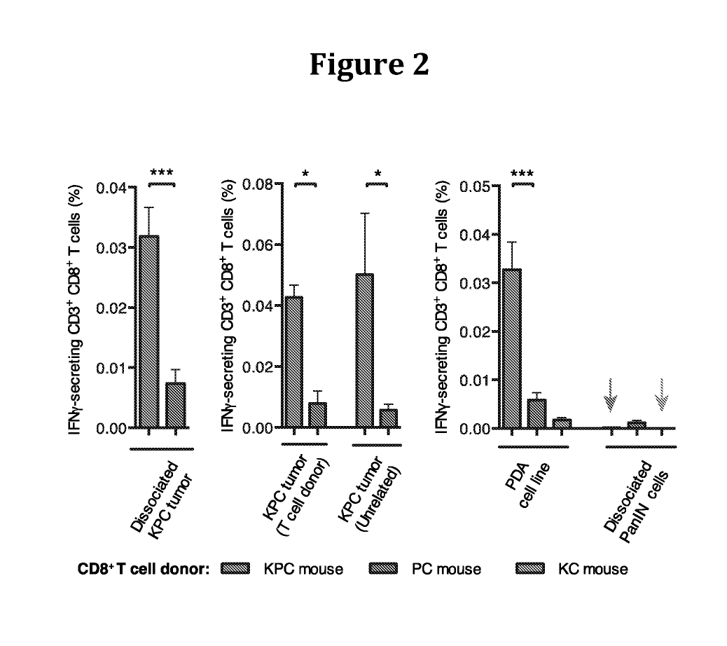

We examined whether blocking immunological checkpoints with α-CTLA-4 and α-PD-L1 would promote immune control of the tumor. Administering α-CTLA-4 or α-PD-L1 antibodies over 6 days to mice bearing PDA did not diminish the ˜80% increase in tumor volume in mice receiving control IgG (FIG. 1). As in humans with PDA, these antibodies were without effect on the rate of tumor growth. We determined whether this could be explained by the absence of an immune response to the PDA. Splenic CD8+ T cells from PDA-bearing and non-tumor-bearing mice were stimulated with tumor cells, and interferon (IFN)-γ-secreting CD8+ T cells reporting antigenic stimulation were detected in an ELISpot assay. Tumor cells induced more ELISpots among CD8+ T cells from tumor-bearing mice than from non-tumor-bearing mice (FIG. 2 left). The frequency of IFN-γ-secreting CD8+ T cells was the same when the stimulating tumor cells were from the T cell donor or from another PDA-bearing mouse (FIG. 2 middle). An established PDA cell line also was stimulatory, whereas dissociated cells from pancreata of KC (LSL-KrasG12D/+;Pdx-1-Cre) mice with pre-malignant pancreatic intraepithelial neoplasia (PanIN) expressing only KrasG12D, or from young KPC mice before the development of cancer, were not (FIG. 2 right). Therefore, PDA bearing mice have a spontaneous adaptive immune response to antigens that is shared by cancer cells from different PDA tumors, and the ineffectiveness of α-CTLA-4 and α-PD-L1 provides indication of an additional immunosuppressive mechanism.

PDA was excised from KPC mice, single cells prepared by enzymatic digestion and CD11b+ (n=4), CD3ε+ (n=1), FAP+ (n=3) and PanIN/PDA (CD11b−/CD3−/FAP−) (n=4) were isolated by FACS. Cxcr4 and Tbp mRNA were measured in the sorted populations by qRT-PCR.

FAP+ stromal cells were observed by immunofluorescent confocal microscopy to be present in PanIN and both cytokeratin-19(CK19)+ and CK19− PDA lesions. FAP+ cells in PanIN were found to express CD34 but rarely α-smooth muscle actin (αSMA), whilst FAP+ cells amongst PDA cells were CD34− and αSMA+.

All FAP+ cells were PDGFRα+ and CD45−, confirming their mesenchymal origin. CD45−/FAP+ stromal cells in enzyme-dispersed single cell suspensions of PDA tumors were enumerated by FACS. Their frequency among dispersed tumor cells was 3.7% (95% CI, 1.1-6.3%; n=8). The expression of FAP by 92% of the αSMA+ fibroblasts (95% CI 87.5-97.1%; n=5) suggested that they are carcinoma-associated fibroblasts (CAFs), which was corroborated by the transcriptomes of CD34+ and CD34− FAP+ cells exhibiting the “inflammatory gene signature” of CAFs (11), as demonstrated by a heat map presenting the RPKM of RNA-Seq analyses of FACS-purified FAP+ cells.

FAP cells from three normal tissues also displayed the signature and were clustered together by a principle component analysis of their transcriptomes, suggesting that FAP may identify a stromal cell lineage. PDA-associated FAP+/CD34− cells may be distinct from other FAP+ subsets.

The expression by tumoral FAP+ cells of genes encoding extracellular matrix proteins verifies their role in the desmoplasia of PDA, whilst their lower expression of decorin may be relevant as this proteoglycan is reported to suppress cancer growth.

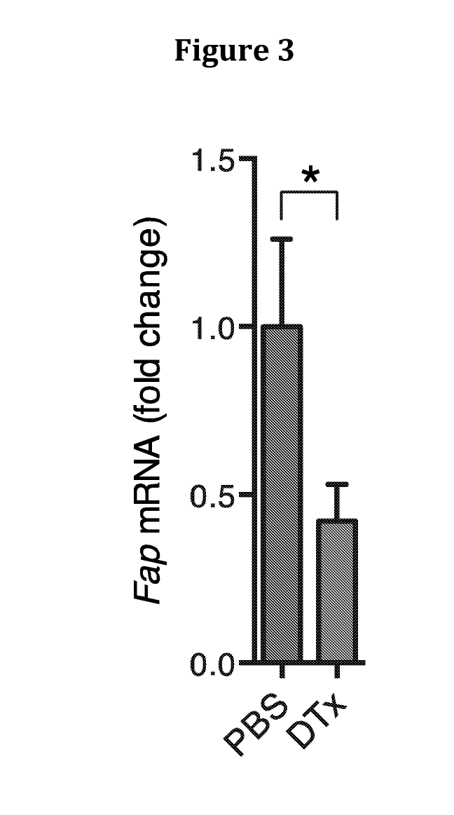

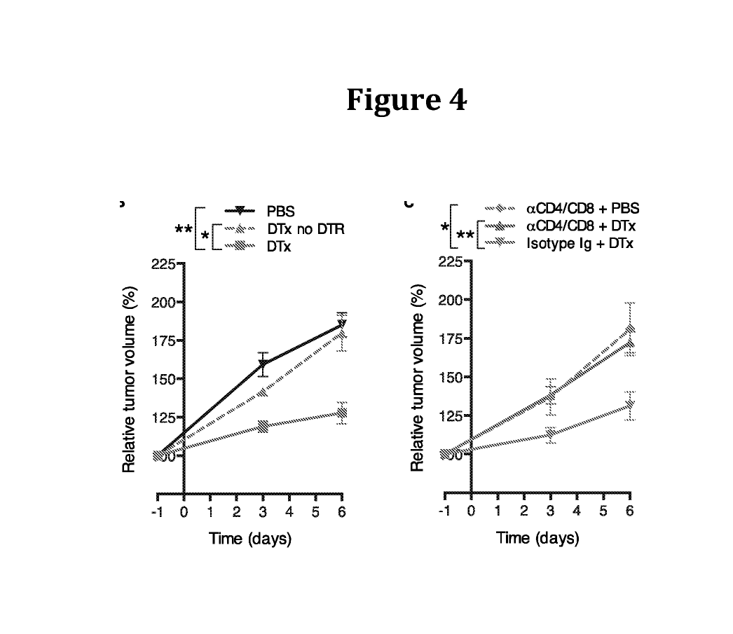

We introduced into the KPC line a bacterial artificial chromosome (BAC) transgene containing a modified Fap gene that drives the expression of the human diphtheria toxin receptor (DTR) selectively in cells that are FAP+. Administering diphtheria toxin (DTx) to PDA-bearing BAC transgenic mice depleted the tumoral FAP+ cell content by approximately 55% (FIG. 3). Depleting FAP+ cells slowed PDA growth (FIG. 4 left), but not when CD4+ and CD8+ T cells were removed (FIG. 4 right), indicating that the observed effect is T-cell dependent.

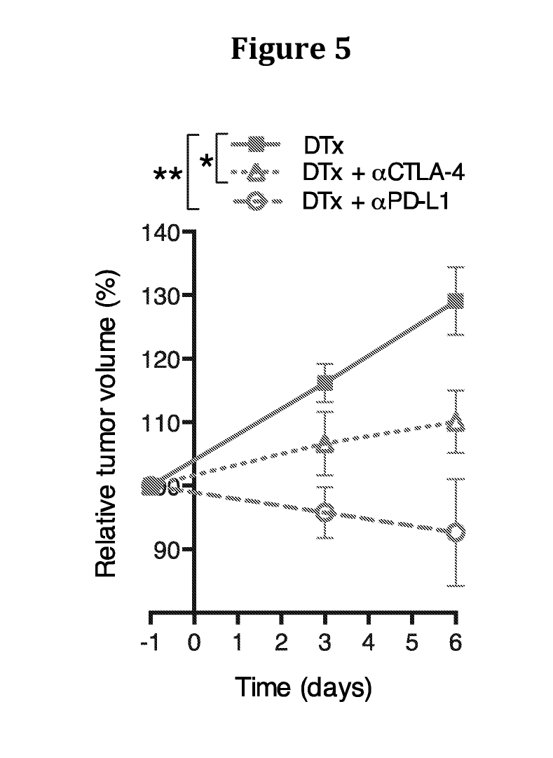

Combining depletion of FAP+ cells with administration of α-CTLA-4 or α-PD-L1 further diminished tumor growth (FIGS. 5 & 6), providing indication that the FAP+ cell contributes to the resistance of murine PDA to these checkpoint antagonists.

The induction by the K8484 PDA cell line of IFN-g-secretion by purified splenic CD8+ T cells from various donors was measured by ELISpot assay. 1 DTx+α-PD-L1 n=2; pre-tumor KPC n=4; all other groups n≧6. The absence of an increase in IFN-γ-secreting CD8+ T cells from the spleens of DTx- and α-PD-L1-treated mice indicates that immune control was not accomplished by enhanced priming of cancer specific CD8+ T cells.

### Example 2

### The Activity of FAP+ Cells is Mediated by CXCL12

Therapy involving the depletion of FAP+ cells is precluded by their essential roles in normal tissues (12), and a therapeutic target that accounts for their immunosuppression must be identified. We noted from immunofluorescent confocal microscopy, that there was a paucity of CD3+ T cells, but not CD11b+ myelomonocytic cells, in the vicinity of cancer cells, a characteristic also of human PDA that is associated with FAP+ cells and other carcinomas (14, 15).

This T cell trafficking problem directed attention to the chemokine, CXCL12, which was observed by confocal immunofluorescent microscopy to localize to cancer cells in both human (13) and murine PDA.

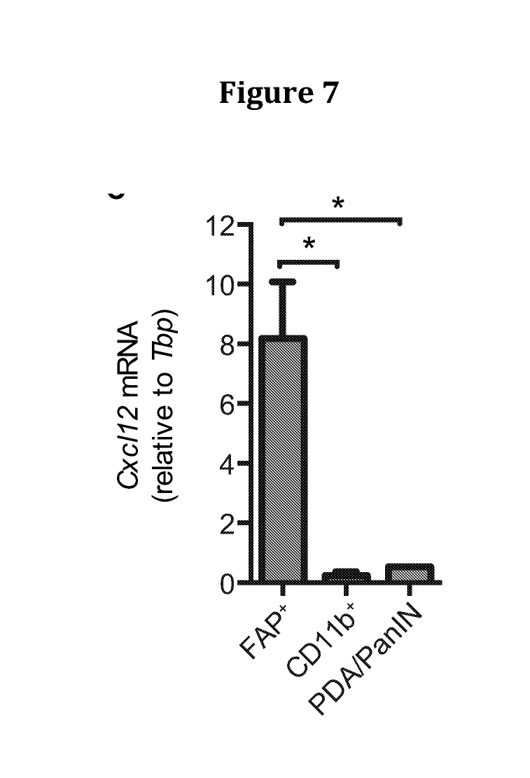

We identified the source of CXCL12 as the tumoral FAP+ cell (FIG. 7), as has been previously reported for CAFs (16).

LL2/OVA tumors were excised from C57BL/6 mice, single cell suspensions prepared by enzymatic digestion, stained with antibodies to FAP, CD45, CD31, and Thy1.1 (for LL2/OVA cells), and isolated by FACS. Cxcl12 and Tbp mRNA were measured in the sorted populations by qRT PCR. FAP+ cells in the subcutaneous Lewis lung carcinoma (LL2) model were found to be the tumoral source of CXCL12.

PDA was excised from KPC mice, single cells prepared by enzymatic digestion and CD11b+ (n=4), CD3ε+ (n=1), FAP+ (n=3) and PanIN/PDA (CD11b−/CD3−/FAP−) (n=4) were isolated by FACS. Cxcr4 and Tbp mRNA were measured in the sorted populations by qRTPCR. CXCR4, the CXCL12 receptor, is unlikely to mediate the uptake of the chemokine because cancer cell expression of CXCR4 was found to be low. We hypothesize that HMGB1 is overexpressed and secreted by cancer cells, and forms a heterocomplex with the chemokine (17).

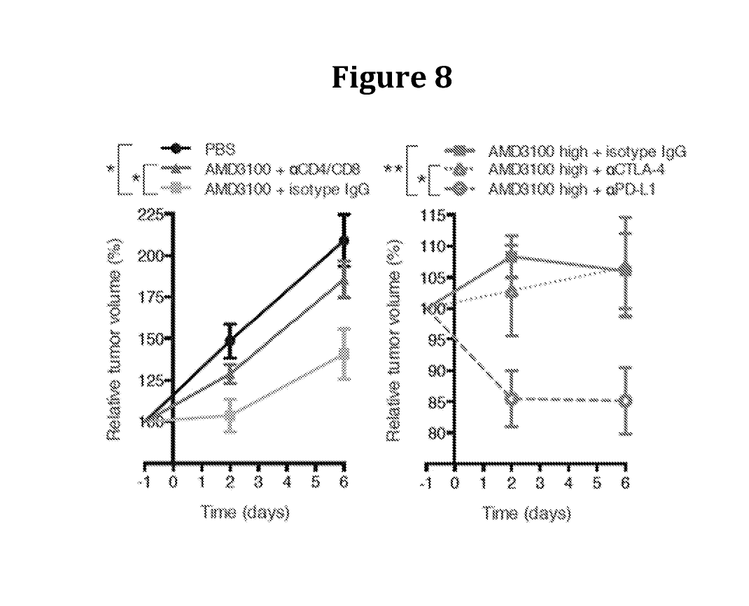

To assess the role of CXCL12 in tumoral immunosuppression, we administered AMD3100 (1,1′-[1,4-Phenylenebis(methylene]bis[1,4,8,11-tetraazacyclotetradecane]), a specific CXCR4 inhibitor (18), to mice bearing PDA in the presence or absence of depleting antibodies to CD4+ and CD8+ T cells. Tumor growth was slowed by AMD3100 in a T cell-dependent manner (FIG. 8 left).

Continuous delivery osmotic pumps containing PBS or AMD3100 (30 mg/ml) were implanted in C57BL/6 and Rag2−/− C57BL/6 mice bearing established, subcutaneous LL2/OVA tumors, and tumor volumes were measured by ultrasound (n=5 for all groups). AMD3100 was observed to induce T cell-dependent control of subcutaneous immunogenic LL2 tumors.

We administered a higher dose of AMD3100 that almost completely arrested PDA growth.

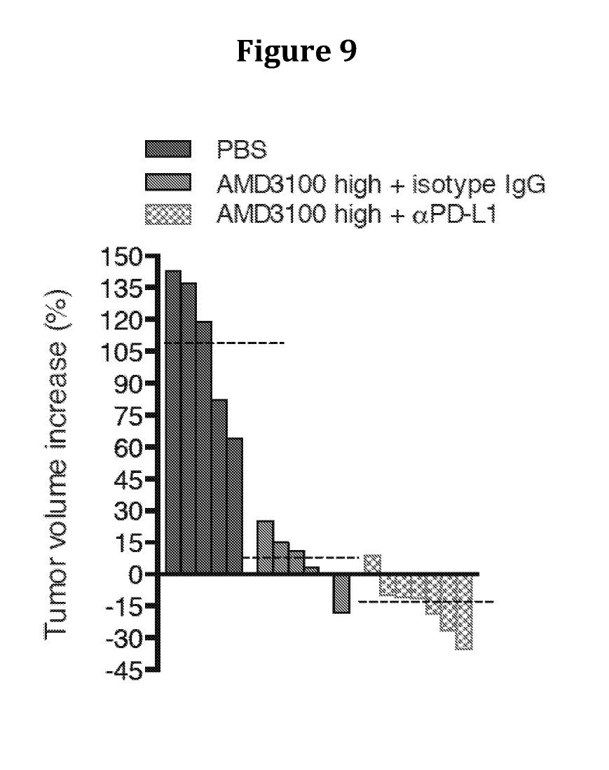

We administered a higher dose of AMD3100 that almost completely arrested PDA growth, and combined it with immunological checkpoint antagonists (FIG. 8 right, FIG. 9). The combination with α-PD-L1 led to a significant 15% decline in PDA volume by 48 h that was maintained for 6 days. In 6 of 7 mice (all but mouse MH16306) reduced tumor size by day 6, relative to tumor size at day −1 (FIG. 9). A reduction in the volume of a PDA tumor, with a protocol in which mice are treated only when tumor sizes reach 200 mm3 (the standard approach in the CRI), has not been previously observed in the KPC model.

In contrast, α-CTLA-4 did not augment the antitumor effect of AMD3100.

To determine if AMD3100 enhanced T cell infiltration amongst cancer cells, we treated mice for 24 h with PBS, AMD3100, α-PD-L1 or both AMD3100 and α-PD-L1. α-PD-L1 alone had no effect, whilst AMD3100 increased the accumulation of T cells, and the combination of α-PD-L1 with AMD3100 amplified this effect, and decreased the frequency of p53+ LOH cells.

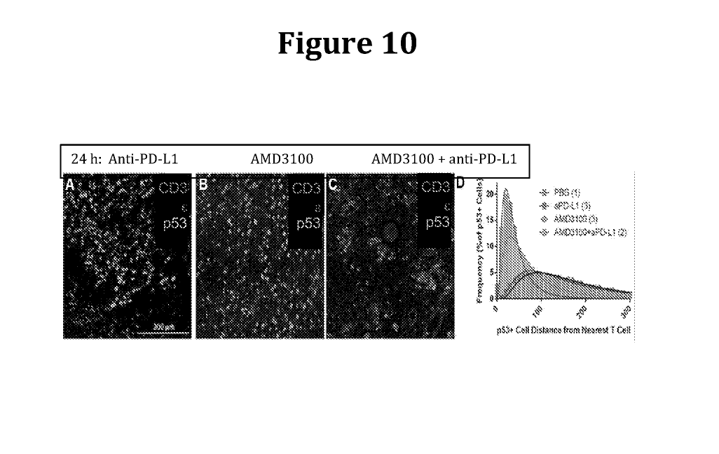

Twelve KPC tumors were taken from mice 24 h after initiating treatment with PBS, α-PD-L1, AMD3100 high, AMD3100 high+α-PD-L1 (n=3 per group), respectively, and assessed for p53 and CD3 by confocal microscopy. AMD3100 and α-PD-L1 were observed to induce the accumulation of CD3+ T cells in cancer-cell-containing regions of PDA (See, FIG. 10.)

KPC tumors were taken from mice 24 h after initiating treatment with PBS and AMD3100 high+α-PD-L1, respectively, were assessed for FoxP3 by immunofluorescent confocal microscopy. Since Foxp3+ cells were observed to increase in the tumors of mice given AMD3100 and α-PD-L1, we concluded that regulatory T cells are not involved in immunosuppression.

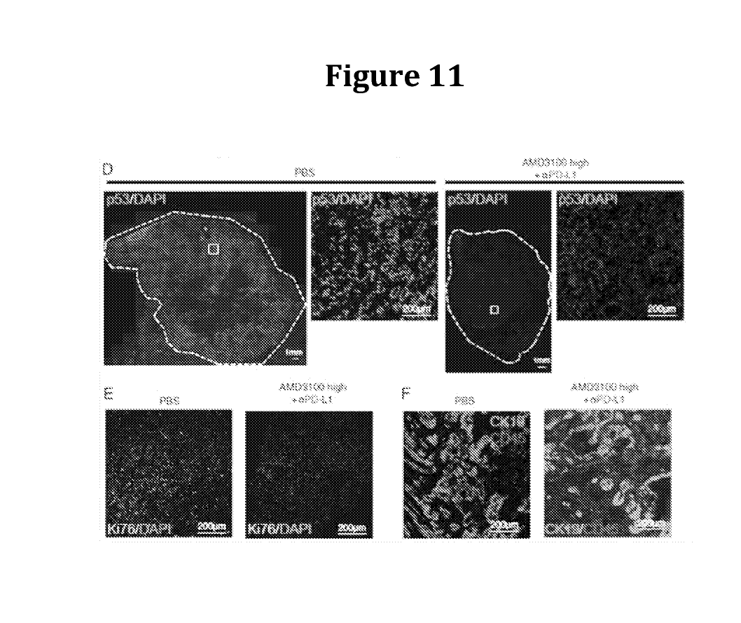

We examined tumors after 6 days of treatment for the presence of p53+ LOH cancer cells using immunofluorescent confocal microscopy and ARIOL scanning p53+ LOH cancer cells were abundant in tumors from mice that had received PBS or α-PD-L1, but were rare in tumors from the mice that had received AMD3100 either alone or with α-PD-L1. (See FIG. 11, panel D). A marked decrease of cancer cells was confirmed using immunofluorescent confocal microscopy by a diminution in proliferating Ki67+ cells (FIG. 11, panel E).

The residual tumors after treatment with AMD3100 alone or with α-PD-L1 were found by ARIOL scanning to be comprised of premalignant CK19+ epithelial cells and CD45+ inflammatory cells (FIG. 11, panel F). The selective elimination of p53+ LOH PDA is consistent with the finding that the CD8+ T cell response is specific for cancer cells (FIG. 2 right).

These studies reveal a hierarchy of immunosuppression in murine PDA, since depleting FAP+ stromal cells or inhibiting the interaction of their chemokine, CXCL12, with CXCR4 uncovers the anti-tumor activity of immunological checkpoint antagonists. We do not know whether CXCR4-mediated exclusion of T cells reflects T cell apoptosis, as occurs with HIV gp120 (19), or a chemo-exclusion effect of CXCL12 (20). The observed absence of a synergistic interaction between AMD3100 and α-CTLA-4 may indicate that inhibition of CXCR4 so effectively promotes the accumulation of T cells among cancer cells that any augmented T cell priming by α-CTLA-4 is superfluous, or that synergy was not observed in this particular assay. This would be consistent with α-CTLA-4 having an effect when the source of the CXCL12, the FAP+ cell, could be only partially depleted with DTx. The remarkable synergistic interaction of AMD3100 with α-PD-L1 indicates that the principal, overarching immunosuppressive process in murine PDA is the limited access of effector T cells to the cancer cells. Since T cells are also excluded from other carcinomas (14, 15), these findings may be widely relevant to tumor immunotherapy.

In the present study, we demonstrate that the autochthonous KPC (LSL-KrasG12D/+; LSL-Trp53R172H/+;Pdx-1-Cre) model of PDA (10) replicates the resistance of human PDA to checkpoint antagonists, despite the presence of systemic anti-PDA immunity. This failure of immunosurveillance is attributable to local immunosuppression mediated by the FAP+ stromal cell, which manifests as exclusion of T cells from regions of the tumor containing PDA cells and involves its production of CXCL12. Inhibiting CXCR4, the CXCL12 receptor, promotes T cell infiltration and synergizes with the checkpoint antagonist, α-PD-L1, to cause cancer regression.

The findings set out in this application have identified both the overarching immune suppressive process in PDA that accounts for the lack of efficacy of anti-PD-L1 in human and murine PDA, and the means for therapeutically reversing it, providing indication that blockade of PD-1 signalling may be efficacious in reducing immunosuppression in combination with blockade of CXCR4 signalling.

### REFERENCES

1. D. Leach et al Science 271, 1734-1736 (1996).
2. H. Dong et al Nat Med. 8, 793-800 (2002).
3. F. S. Hodi et al N. Engl. J. Med. 363, 9 711-23 (2010).
4. O. Hamid et al N. Engl. J. Med. 369, 134-12 44 (2013).
5. J. R. Brahmer et al N. Engl. J. Med. 15 366, 2455-65 (2012).
6. J. D. Wolchok et al N. Engl. J. Med. 369, 122-33 (2013).
7. R. E. Royal et al J. Immunother. 33, 828-33 (2010).
8. M. Kraman et al. Science. 330, 827-30 (2010).
9. P. Garin-Chesa, et al Proc. Natl. Acad. Sci. USA. 87, 7235-9 (1990).
10. S. R. Hingorani et al Cancer Cell. 7, 469-83 (2005).
11. N. Erez et al Cancer Cell. 17, 135-47 (2010).
12. E. W. Roberts, et al J. Exp. Med. 210, 1137-51 (2013).
13. J. J. Liang et al Cancer Epidemiol. Biomarkers Prev. 19, 2598-604 (2010).
14. Y. Naito et al Cancer Res. 58, 3491-4 (1998).
15. L. Zhang, et al. N. Engl. J. Med. 348, 203-13 (2003).
16. A. Orimo et al. Cell. 121, 335-17 48 (2005).
17. M. Schiraldi et al J. Exp. Med. 209, 551-63 (2012).
18. D. Schols et al Antiviral Res. 35, 147-56 (1997).
19. G. Herbein et al. Nature. 395, 189-94 (1998).
20. M. C. Poznansky et al. Nat. Med. 6, 543-8 (2000).
21. G. Wei et al Immunity. 35, 299-311 (2011).
22. Righi et al., Cancer Res. 2011 Aug. 15; 71(16):5522-34)
23. Vianello et al., J Immunol. 2006 Mar. 1; 176(5):2902-14
24. Nomura et al., Int J Cancer. 2001 Mar. 1; 91(5):597-606
25. Fushimi et al., Cancer Res. 2006 Apr. 1; 66(7):3513-22
26. Williams et al., Mol Cancer. 2010 Sep. 17; 9:250
27. Dannussi-Joannopoulos et al., Blood. 2002 Sep. 1; 100(5):1551-8

1-44. (canceled)

45. A method of treating a patient suffering from cancer, wherein the method comprises administering to the patient a CXCL12 antagonist and a checkpoint antagonist.

46. The method of claim 45, wherein the CXCL12 antagonist is selected from:
a) an anti-CXCL12 antibody;
b) RNA oligonucleotide NOX-A12; and
c) Tannic acid.

47. The method of claim 45, wherein the checkpoint antagonist acts synergistically with the CXCL12 antagonist.

48. The method of claim 45, wherein the checkpoint antagonist is a PD-1 antagonist or a PD-L1 antagonist.

49. The method of claim 48, wherein the PD-1 antagonist or the PD-L1 antagonist is selected from:
a) an anti-PD-1 antibody; or
b) an anti-PD-L1 antibody.

50. The method of claim 45, wherein the checkpoint antagonist is a CTLA-4 antagonist, TIM-3 antagonist, or a LAG3 antagonist.

51. The method of claim 45, wherein said method inhibits T cell exclusion in a tumor.

52. The method of claim 45, wherein said method increases the proximity or the frequency of the T-cells among the cancer cells contained in the tumor.

53. The method of claim 52, wherein the T-cells are selected from CD3+ T-cells or CD3+ effector T-cells.

54. The method of claim 45, wherein the CXCL12 antagonist and the checkpoint antagonist is administered either simultaneously or separately.

55. The method of claim 45, wherein the method increases the sensitivity of the cancer cells to the host immune responses.

56. The method of claim 45, wherein the method reduces immune suppression in the tumor.

57. The method of claim 45, wherein cancer cell immune recognition is increased within the tumor.

58. The method of claim 45, wherein cancer cell growth is inhibited or reduced.

59. The method of claim 45, wherein the tumor is resistant to immunotherapy.

60. A method of treating a patient suffering from cancer, wherein the method comprises administering to the patient a CXCR4 antagonist and a checkpoint antagonist.

61. The method of claim 60, wherein the CXCR4 antagonist is selected from:
a) an anti-CXCR4 antibody;
b) BMS-936564/MDX-1338;
c) LY2510924;
d) 1,1′-[1,4-phenylenebis(methylene)]bis[1,4,8,11-tetraazacyclotetradecane] (AMD3100; Plerixafor);
e) N,N-dipropyl-N-[4-({[(1H-imidazol-2-yl)methyl)benzyl][(1-methyl-1H-imidazol-2-yl)methyl]amino]methyl)benzyl]-N-methylbutane-1,4-diamine tri(2R,3R)-tartrate (KRH-3955);
f) ([5-(4-methyl-1-piperazinyl)-2-({methyl[(8S)-5,6,7,8-tetrahydro-8-quinolinyl]amino}methyl)imidazo[1,2-a]pyridin-3-yl]methanol) (GSK812397); or
g) N-(1H-benzimidazol-2-ylmethyl)-N′-(5,6,7,8-tetrahydroquinolin-8-yl)butane-1,4-diamine (AMD11070).

62. The method of claim 60, wherein the checkpoint antagonist acts synergistically with the CXCR4 antagonist.

63. The method of claim 60, wherein the checkpoint antagonist is a PD-1 antagonist or a PD-L1 antagonist.

64. The method of claim 63, wherein the PD-1 antagonist or the PD-L1 antagonist is selected from:
a) an anti-PD-1 antibody; or
b) an anti-PD-L1 antibody.

65. The method of claim 60, wherein the checkpoint antagonist is a CTLA-4 antagonist, TIM-3 antagonist, or a LAG3 antagonist.

66. The method of claim 60, wherein said method inhibits T cell exclusion in a tumor.

67. The method of claim 60, wherein said method increases the proximity or the frequency of the T-cells among the cancer cells contained in the tumor.

68. The method of claim 67, wherein the T-cells are selected from CD3+ T-cells or CD3+ effector T-cells.

69. The method of claim 60, wherein the CXCR4 antagonist and the checkpoint antagonist is administered either simultaneously or separately.

70. The method of claim 60, wherein the method increases the sensitivity of the cancer cells to the host immune responses.

71. The method of claim 60, wherein the method reduces immune suppression in the tumor.

72. The method of claim 60, wherein cancer cell immune recognition is increased within the tumor.

73. The method of claim 60, wherein cancer cell growth is inhibited or reduced.

74. The method of claim 60, wherein the tumor is resistant to immunotherapy.
